(19) United States
(12) Patent Application Publication
(10) Pub. No.: US 2010/0028312 A1
(43) Pub. Date: Feb. 4, 2010

(54) Stably transformed bone marrow-derived cells and uses thereof

(75) Inventors: Ryuichi Aikawa, Brookline, MA (US); Douglas W. Losordo, Chicago, IL (US)

Correspondence Address: EDWARDS ANGELL PALMER & DODGE LLP, P.O. BOX 55874, BOSTON, MA, 02205, US

(73) Assignee: Caritas St. Elizabeth Medical Center of Boston Inc, Boston, MA (US)

(21) Appl. No.: 11/887,021

(22) PCT Filed: Mar. 24, 2006
(86) PCT No.: PCT/US06/10981

§ 371 Date: May 22, 2009

Related U.S. Application Data
(60) Provisional application No. 60/665,431, filed on Mar. 24, 2005.
(60) Provisional application No. 60/673,305, filed on Apr. 19, 2005.
(60) Provisional application No. 60/735,572, filed on Nov. 10, 2005.

(51) Int. Cl. A61K 35/12

(52) U.S. Cl. 424/93.21

(57) ABSTRACT

The invention provides compositions comprising genetically modified bone marrow cells and related therapeutic and diagnostic methods. Transduced bone marrow cells can be therapeutically administered to a subject, such as a human patient to provide for the expression of an encoded protein in the subject in need thereof.

### CROSS-REFERENCE TO RELATED APPLICATION

This application claims the benefit of the following U.S. Provisional Application Nos. 60/665,431, which was filed on Mar. 24, 2005; 60/673,305, which was filed on Apr. 19, 2005; and 60/735,572, which was filed on Nov. 10, 2005; the disclosures of which are hereby incorporated by reference in their entirety.

### FIELD OF THE INVENTION

The invention generally relates to compositions and methods useful for cell-mediated therapy using isolated bone marrow (BM) cells. In one embodiment, the invention relates to the manufacture and use of genetically modified BM cells to prevent, treat or reduce the severity of cardiac ischemia.

### BACKGROUND

Recombinant AAV (rAAV) have been used for the delivery and long-term expression of genes in animal cells, including clinically important non-dividing cells of the brain, liver, skeletal muscle and lung. Clinical trials using this technology have included use of rAAV expressing the cftr gene as a treatment for cystic fibrosis.

Traditionally, therapeutic vectors have been introduced to the tissue of a subject directly, for example by inhalation of vectors for pulmonary gene therapy, or by injection of vector DNA into tissues where vector expression is required. Recently there has been recognition that cellular systems provide an attractive alternative for delivery of therapeutic gene products to tissues or cells in need. In particular, there is considerable interest in the use of genetically modified stem cells to serve as vehicles for delivery of gene products of therapeutic interest to target tissues.

Recombinant adeno-associated virus (rAAV) are used to transfer genes of interest into cells where gene expression is desired. The small size and physical stability of rAAV make these vectors advantageous for in vivo use, and transgene expression f can persist over the long term in diverse tissues, including heart and skeletal muscle. Advantageously, undesirable side effects, such as inflammation, have not been reported for these vectors in in vitro and in vivo experiments. rAAV vectors have the capacity to transduce not only non-dividing cells, but also dividing cells

Hematopoietic stem cells of the erythroid lineage and bone-forming mesenchymal stem cells are a rich and varied source of stem cells with remarkably diverse capabilities to renew themselves and produce progeny cells committed to divide and differentiate along myriad lineages, including cells of the blood, cardiovascular system, nervous system, liver, kidney and other tissues.

It would be desirable to have long-lasting and multi-lineage stem cells derived from bone marrow (BM) that are capable of expressing gene products of therapeutic interest.

### SUMMARY OF THE INVENTION

The invention provides compositions comprising a genetically modified hematopoietic stem cell (e.g., a bone marrow (BM)-derived cell or a cord blood derived stem cell) and related therapeutic and diagnostic methods.

In one aspect, the invention generally features a method for expressing a therapeutic or reporter gene in a cardiac tissue or a blood vessel of a host subject. The method involves contacting a hematopoietic stem cell with a recombinant adeno-associated viral vector containing a nucleic acid sequence encoding a therapeutic (e.g., IGF-1 or human growth hormone) or reporter polypeptide (e.g., β-galactosidase, glucuronidase (GUS), luciferase, and chloramphenicol transacetylase (CAT)) to obtain a transgenic cell stably transduced with the vector; and administering the cell to a host subject, such that the transgenic cell or a progeny cell thereof populates a cardiac tissue or a blood vessel in the subject and expresses the therapeutic or reporter polypeptide.

In another aspect, the invention provides a method for expressing an IGF-1 polypeptide in a cardiac tissue of a host subject in need thereof. The method involves contacting a hematopoietic stem cell with an expression vector containing a nucleic acid sequence encoding an IGF-1 polypeptide to obtain a transgenic cell stably transduced with the vector; and administering the cell to a host subject, such that the cell or a progeny cell thereof populates a cardiac tissue in the subject and expresses an IGF-1 polypeptide.

In yet another aspect, the invention features a method for expressing an IGF-1 polypeptide in a blood vessel of a host subject in need thereof. The method involves contacting a hematopoietic stem cell with an expression vector containing a nucleic acid sequence encoding an IGF-1 polypeptide to obtain a cell stably transduced with the vector; and administering the cell to a host subject, such that the cell or a progeny cell thereof populates a blood vessel in the subject and expresses an IGF-1 polypeptide.

In yet another aspect, the invention features a method for preventing, treating or reducing the severity of a cardiac indication (e.g., cardiac ischemia, myocardial infarction, cardiomyopathy, cardiomyositis, and heart failure) in a host subject in need thereof. The method involves administering to the subject a therapeutically effective amount of a recombinant cell containing a recombinant adeno-associated viral vector encoding a therapeutic polypeptide (e.g., IGF-1 or human growth hormone).

In yet another aspect, the invention features a method for reducing apoptosis in a cardiac tissue. The method involves administering to the subject a therapeutically effective amount of a recombinant cell containing a recombinant adeno-associated viral vector containing a nucleic acid sequence encoding a therapeutic polypeptide.

In yet another aspect, the invention features a method for increasing proliferation in a cardiac tissue. The method involves administering to the subject a therapeutically effective amount of a recombinant cell containing a recombinant adeno-associated viral vector containing a nucleic acid sequence encoding a therapeutic polypeptide.

In yet another aspect, the invention features a method for increasing angiogenesis in a cardiac tissue. The method involves administering to the subject a therapeutically effective amount of a recombinant cell containing a recombinant adeno-associated viral vector containing a nucleic acid sequence encoding a therapeutic polypeptide.

In yet another aspect, the invention features a transgenic cell derived from bone marrow, the cell containing a recombinant adeno-associated viral vector containing a nucleic acid sequence encoding a therapeutic polypeptide (e.g., IGF-1 or human growth hormone) or detectable reporter (e.g., glucuronidase (GUS), luciferase, chloramphenicol transacetylase (CAT), and beta-galactosidase), or functional fragments thereof.

In yet another aspect, the invention features a transgenic cell derived from bone marrow or umbilical cord blood containing a recombinant adeno-associated viral vector containing a nucleic acid sequence encoding an IGF-1 polypeptide, a human growth hormone polypeptide, or a functional fragment thereof. In one embodiment, the cell expresses IGF-1 for at least 8 weeks following administration of the cell to a host subject. In another embodiment, the cell is selected from the group consisting of a progenitor cell of the bone marrow, a cell derived from a transduced stem or progenitor cell of the bone marrow, an endothelial progenitor cell (EPC), a hematopoietic stem cell (HSC), a mesenchymal stem cell (MSC), a multipotent adult progenitor cell (MAPC) and a human multipotent bone marrow stem cell. In yet another embodiment, the cell is a human multipotent stem cell having a reduced level of a marker selected from the group consisting of CD90, CD117, CD34, CD113, FLK-1, tie-2, Oct 4, GATA-4, NKx2.5, Rex-1, CD105, CD117, CD133, MHC class I receptor and MHC class II receptor. In yet another embodiment, the cell expresses reduced levels of at least two, three, four, or more markers or of all markers. In another embodiment, the reduced marker expression is undetectable in a standard cell marker detection assay. In another embodiment, the cell is stably transduced.

In yet another aspect, the invention provides a graft containing a transgenic cell of any previous aspect.

In yet another aspect, the invention provides a pharmaceutical composition for preventing, treating or reducing the severity of a heart or vascular disorder in a subject in need thereof, the composition containing a transgenic cell containing a recombinant adeno-associated viral vector containing a nucleic acid sequence encoding a therapeutic or reporter polypeptide. In another embodiment, the cell is present in a graft. In another embodiment, the composition comprises an additional angiogenic factor or functional fragment thereof. In another embodiment, the composition further comprises a nucleic acid encoding an additional angiogenic factor (e.g., VEGF) or functional fragment thereof.

In yet another aspect, the invention provides a kit for transducing a hematopoietic stem cell, the kit containing an adeno-associated viral vector containing a nucleic acid sequence encoding a therapeutic polypeptide or detectable reporter. In one embodiment, the kit further comprises directions for administering the vector to a bone marrow derived cell.

In various embodiments of any of the above aspects, the transduced cell is a hematopoietic stem cell (e.g., bone marrow derived cell or cell derived from cord blood) isolated and expanded in vitro to obtain a cell population enriched in bone marrow-derived stem or progenitor cells stably transduced with the vector prior to being administered to the host subject. In various embodiments of any of the above aspects, IGF-1 is expressed in the cell for at least 2, 4, 6, 8, 12, 16, 20, 24, 32, 40 or 60 weeks following administration of the cell to a host subject. In various embodiments of any of the above aspects, the cell is selected from the group consisting of a progenitor cell of the bone marrow, a stem cell of the bone marrow, an endothelial progenitor cell (e.g., EPC is isolated from bone marrow or peripheral blood of a donor subject), a hematopoietic stem cell (HSC), a mesenchymal stem cell (e.g., a cell derived from cord blood or a human umbilical cord), a multipotent adult progenitor cell, and a human multipotent bone marrow stem cell. In various embodiments of any of the above aspects, the cell is treated with a tyrosine kinase inhibitor (e.g., genistein) prior to infection with an adeno-associated viral vector. In various embodiments of any of the above aspects, the human stem cell having reduced levels of a marker selected from the group consisting of: CD90, CD117, CD34, CD113, FLK-1, tie-2, Oct 4, GATA-4, NKx2.5, Rex-1, CD105, CD117, CD133, MHC class I receptor and MHC class II receptor. In yet other embodiments of any of the above aspects, the cell expresses reduced levels of at least two, three, four, or more markers, such as reduced levels of all markers or the marker expression is undetectable in a standard cell marker detection assay. In various embodiments of any of the above aspects, the method further containing administering to the host subject an angiogenic factor or a nucleic acid encoding an angiogenic factor. In various embodiments of any of the above aspects, the angiogenic factor is VEGF, IGF-1, or a functional fragment thereof. In yet other embodiments, the stably transduced cell (e.g., derived from a donor subject) is administered parenterally, by bone marrow transplantation, by direct injection into a cardiac tissue or via a blood vessel supplying the heart. In still other embodiments, the donor subject and the host subject are the same individual. In yet other embodiments, the host subject is diagnosed as having a cardiac indication selected from the group consisting of cardiac ischemia, myocardial infarction, cardiomyopathy, cardiomyositis, and heart failure.

In various embodiments of any of the above aspects, the vector is a replication defective adeno-associated viral vector (e.g., a vector that has an AAV serotype of AAV-1, AAV-2, AAV-3, AAV-4, AAV-5, AAV-6, AAV-7 and AAV-8). In various embodiments of any of the above aspects, the stem cell is in vitro or in vivo. In still other embodiments of any of the above aspects, the cell or a progeny cell thereof can populate the spleen, the peripheral blood, or a cardiac tissue or a blood vessel in a host subject. In various embodiments of any of the above aspects, the cell is a human or rodent cell.

The invention has a wide spectrum of uses including providing transduced hematopoietic stem cells (e.g., bone marrow cells, cord blood cells) that can be therapeutically administered to a subject, such as a human patient. In particular embodiments, such administered cells provide for the relatively long-term expression of an encoded protein in the subject. Uses of the invention include the prevention, treatment or reduction of the severity of a cardiac indication, including but not limited to cardiac ischemia, cardiomyopathy, myocardial infarction, cardiomyopathy, cardiomyositis, and heart failure.

More particularly, we have discovered that a transgene can be stably integrated into the genome of a hematopoietic stem cell (e.g., bone marrow cells, cord blood cells), including but not limited to various classes of stem and progenitor cells of the bone marrow or from the human umbilical cord. Transgenes carried by the transgenic cells of the invention can be expressed for extended periods in vivo (at least several months) in cells derived from hematopoietic stem cell (e.g., bone marrow cells, cord blood cells) infected with viral vectors, such as recombinant adeno-associated viral vectors (rAAV). The rAAV-infected cells when delivered to a recipient (host) subject by bone marrow transplantation (BMT) can migrate to and populate diverse tissues, such as peripheral blood (PB), bone marrow (BM), spleen, cardiac tissue and blood vessels. By “populate” is meant contribute at least one rAAV infected cell to a tissue or organ.

In certain embodiments of the methods of the invention, administration of an angiogenic factor (e.g., a VEGF protein) is used in conjunction with administration of the cells to significantly increase the rAAV-mediated transgene expression in BM and PB of the recipients. Importantly, in recipient subjects experiencing myocardial infarction, ischemic heart areas exhibited significant transgene expression in subjects receiving transplants of BM infected with rAAV.

Accordingly, the rAAV vectors of the invention are useful for transducing hematopoietic stem cell (e.g., bone marrow cells, cord blood cells) ex vivo prior to introduction into a host, for example by BMT. The compositions comprising transgenic hematopoietic stem cell (e.g., bone marrow cells, cord blood cells) and their methods of use hold great promise for a wide variety therapeutic purposes, including delivery of therapeutic gene products for treatment of cardiovascular disorders. A “therapeutic polypeptide” is a polypeptide or functional fragment thereof that prevents, treats, or ameliorates a pathological condition, such as a cardiac indication, when administered to a subject. Exemplary therapeutic polypeptides include IGF-1 and human growth hormone.

Accordingly, one important aspect of the invention is an isolated transgenic cell derived from bone marrow or an isolated transgenic cell derived from cord blood. The cell is stably transduced with an expression vector comprising a nucleic acid sequence encoding a therapeutic or reporter gene. By “reporter gene” is meant a gene encoding a gene product whose expression may be assayed; such polypeptides include, without limitation, glucuronidase (GUS), luciferase, chloramphenicol transacetylase (CAT), and beta-galactosidase.

The invention encompasses a wide variety of hematopoietic stem cell (e.g., bone marrow cells, cord blood cells) and BM-derived cell types, including stem or progenitor cells of the bone marrow. Because the transgene is stably integrated into the genome of the cell, the transgenic cells include those cells that are derived from a transduced stem or progenitor cell of the bone marrow. Thus, a transgenic stem or progenitor cell of the invention may migrate from the bone marrow to a distant site in the body, and through successive divisions and/or processes of differentiation, transform into another transgenic cell type derived from the stem/progenitor cell. Alternatively, a transgenic cell line can be derived in vitro by propagation of a stably transduced hematopoietic stem cell (e.g., bone marrow cells, cord blood cells).

Specific embodiments of the transgenic cells of the invention are distinguished by expression of particular combinations of cellular markers, (or in some cases the reduction or absence of expression of particular markers). Transgenic hematopoietic stem cell (e.g., bone marrow cells, cord blood cells) of the invention include, but are not limited to, characterized cell types including: endothelial progenitor cells (EPC); hematopoietic cells (HSC) (e.g., bone marrow-derived cells, cord blood cells); mesenchymal stem cells (MSC); multipotent adult progenitor cells (MAPC); and human multipotent bone marrow stem cells (hBMSC) characterized by reduced levels of expression of one or more (e.g., 2, 3, 4, 5, 6, 7, 8, 9, 10, 11, 12, 13, 14, or 15) of the following markers: CD90, CD117, CD34, CD113, FLK-1, tie-2, Oct 4, GATA-4, NKx2.5, Rex-1, CD105, CD117, CD133, MHC class I receptor and MHC class II receptor as determined by standard cell marker detection assay. Expression of these markers may be reduced by 10%, 25%, 50%, 75% or 100% relative to expression in a control cell. Suitable control cells include mononuclear cells that occur in bone marrow or blood. In some embodiments, expression is so low as to be undetectable in standard assays.

Expression of the therapeutic or reporter gene in the transgenic cells can occur for at least 2, 3, 4, 5, 6, 7, 8, 12, 16, 20, or 24 weeks in vivo after administration of the cell to a host subject, or for longer periods.

The cells of the invention can be administered to a subject in need thereof by any suitable route using methods in accord with the invention. For example, the cells can be administered to the bone marrow of the host subject, or to the subject's bloodstream.

In particular applications involving cardiac and cardiovascular disorders, the cell or vector of the invention can be administered directly to the heart or to a blood vessel that supplies the heart of a host subject in need of such treatment.

Upon administration to a host subject, the transgenic cells of the invention are capable of widespread distribution throughout the body. In some embodiments, the transduced cells of the invention populate the spleen, peripheral blood, cardiac tissue or a blood vessel. Also included in the invention in various embodiments are grafts comprising at least one of the isolated transgenic cells of the invention.

In another aspect, the invention provides a method for expressing a therapeutic or reporter gene in a cardiac tissue or a blood vessel. The method includes the steps of: (a) contacting a population of hematopoietic stem cell (e.g., bone marrow cells from a donor subject, cord blood cells) with an expression vector comprising a nucleic acid sequence encoding a therapeutic or reporter gene, to obtain a bone marrow-derived transgenic stem or progenitor cell stably transduced with the vector. The transduced stem or progenitor cell from the cell population of step (a) is typically isolated and expanded in vitro, to obtain a cell population or graft enriched in bone marrow-derived stem or progenitor cells stably transduced with the vector. The transgenic cells are then administered to a host subject, as a cell population or in the form of a graft. Once administered to the subject, the transgenic BM cells or progeny cells derived from them populate a cardiac tissue or a blood vessel of the subject and express the therapeutic or reporter gene therein.

In some embodiments the foregoing method can further include administering at least one angiogenic factor to the host subject by various means. An “angiogenic factor” is any polypeptide or functional fragment thereof that increases, supports or promotes angiogenesis. In one version of the method, at least one nucleic acid encoding at least one angiogenic factor or a functional fragment thereof is administered to the subject.

In certain preferred embodiments of the method, the transgenic cell is an endothelial progenitor cell (EPC). The EPC used for production of the transgenic cells can be isolated from bone marrow of the donor subject, or in some embodiments from the peripheral blood of the donor subject.

The transgenic cells of the invention, e.g., transgenic EPC, can be expanded in vitro and stored frozen until ready for use.

The method can be practiced in various ways, including administration of the cells parenterally or by bone marrow transplantation.

In preferred embodiments of the method, the donor subject and the host subject are the same individual.

In some versions of the method particularly suited to treatment of vascular disorders, transgenic endothelial cells within a blood vessel of the host are derived from the transduced cells.

In other embodiments, cardiac muscle cells in the host subject are derived from the transduced cells.

Another aspect of the invention is a pharmaceutical product for preventing, treating or reducing the severity of a heart or vascular disorder. The product comprises at least one of the following components: the above-described isolated transduced cells, a graft comprising these cells, and optionally directions for preparing, maintaining and/or administering the cells or graft.

The pharmaceutical product can further comprise at least one angiogenic factor or functional fragment thereof. In some embodiments the angiogenic factor can be in the form of at least one nucleic acid encoding an angiogenic factor, or functional fragment thereof.

In preferred pharmaceutical products of the invention, the transduced cells comprise a replication defective adeno-associated viral (AAV) vector. The serotype of the rAAV vector can be any suitable serotype, such as AAV-1, AAV-2, or another available serotype. Examples include AAV-3, AAV-4, AAV-5, AAV-6, AAV-7 or AAV-8.

In preferred embodiments of the foregoing isolated transgenic cells, grafts, or pharmaceutical products, the expression vector comprises a nucleic acid sequence encoding a therapeutic polypeptide that is an angiogenic factor or a hematopoietic factor. One therapeutic polypeptide suitable for use in a method of the invention is IGF-1. An IGF-1 polypeptide is encoded, for example, by GenBank Accession No. X00173; an IGF-1 polypeptide sequence is provided, for example, in GenBank Accession No. CAA24998. Another therapeutic polypeptide useful in the methods of the invention is human growth hormone. The amino acid sequence of human growth hormone is provided, for example, at GenBank Accession No. P01241. The sequence of a nucleic acid molecule encoding a human growth hormone is provided at GenBank Accession No. BC075013.

In yet another aspect of the invention, there is provided a method for preventing, treating or reducing the severity of a coronary disease, such as cardiac ischemia. In one embodiment, the method includes administering to a subject in need of such treatment a therapeutically effective amount of the isolated transduced cells disclosed herein. By “effective amount” is meant an amount sufficient to prevent, treat, or ameliorate a disease or disorder in a subject.

The invention further provides a method of increasing proliferation and decreasing apoptosis of cardiac cells in the heart of a host subject impacted by a coronary disease by administering a therapeutically effective amount of the isolated transduced cells of the invention.

Other aspects and advantages of the invention are discussed below.

### BRIEF DESCRIPTION OF THE DRAWINGS

FIGS. 1A-D show transduction of bone marrow (BM) cells with recombinant AAV vector expressing β-galactosidase. FIG. 1A is a graph showing FACS analysis of LacZ positive cells in peripheral blood (PB) and bone marrow (BM) cells after bone marrow transplantation. FIG. 1B includes six photographs showing β-galactosidase expression in spleen at the macroscopic (a-c) and microscopic (d-f) levels. FIG. 1C is a photograph of a gel showing rAAV-CMV-lacZ PCR product amplified from infected BM cells one month after bone marrow transplantation. FIG. 1D is a graph showing quantitation of lacZ positive cells in rAAV-infected and control BM cells in vitro four weeks after infection.

FIGS. 2A-2B illustrate kinetics of the response of rAAV-infected BM cells to treatment by a cytokine or to ischemic stress. FIG. 2A includes two graphs showing FACS analysis of LacZ expressing cells in peripheral blood (PB, left graph) and bone marrow (BM, right graph) from mice receiving bone marrow transplantation (BMT), with and without treatment with vascular endothelial growth factor (VEGF). FIG. 2B includes three photomicrographs (a-c) and a graph (d) illustrating LacZ expression in the hearts of mice following rAAV infection of BM, bone marrow transplantation and induction of myocardial infarct.

FIGS. 3A-B are two graphs showing mRNA expression of insulin-like growth factor-1 (IGF-1) (3A) and the downstream effector Akt (3B) in cells infected with rAAV vector comprising nucleic acids encoding a reporter gene (lacZ), IGF-1 or growth hormone (GH) according to an embodiment of the invention.

FIG. 4 is a graph depicting the percentage of isolated endothelial progenitor cells (EPC) expressing β-galactosidase activity following transduction with an AAV vector comprising a nucleic acid sequence encoding the β-gal reporter gene, according to an embodiment of the invention.

FIG. 5 is a graph showing enhanced AAV-mediated expression of a reporter gene (β-gal) by isolated EPC pretreated with genistein, an inhibitor of protein tyrosine kinases, according to an embodiment of the invention.

FIG. 6 is a graph showing increased AAV-mediated expression of therapeutic gene IGF-1 by isolated EPC following genistein treatment, according to an embodiment of the invention.

FIGS. 7A-7B are two graphs showing the paracrine effects of EPC transduced with AAV-IGF-1 on cardiomyocyte features, when the tranduced EPC and cardiomyocytes are co-cultured. FIG. 7A shows increased proliferation of cardiomyocytes (brdU expression). FIG. 7B shows decreased apoptosis in these cells, as compared with cardiomyocytes cultured with EPC transduced with control (AAV-LacZ) vector.

FIGS. 8A-8C includes three graphs showing results of cardiac function tests (echocardiography) performed at 0 and 12 weeks in host subjects undergoing myocardial infarction (MI) and transplantation at day 0 of EPC genetically modified to express therapeutic agent IGF-1, or a control gene, LacZ. FIGS. 8A and 8B show changes over time from 0 to 12 weeks post-MI in left ventricular diastolic dimension (LVDd) and left ventricular systolic dimension (LVDs) values, respectively. FIG. 8C shows changes in percentage of fractional shortening.

FIGS. 9A and 9B are two graphs from the study illustrated in FIGS. 8A-8C, showing effects of transplantation of EPC expressing IGF-1 or LacZ (control) at the cellular level in heart tissue from an area impacted by MI, according to an embodiment of the invention. Results at 12 weeks post-MI are illustrated. FIG. 9A shows decreased numbers of apoptotic (TUNEL-positive) cardiac muscle cells in hearts transplanted with EPC transduced with IGF-1, as compared with control vector (LacZ). FIG. 9B shows that proliferation of cardiac muscle cells is increased in subjects receiving autologous transplants of IGF-1 transduced EPC, according to an embodiment of the invention.

### DETAILED DESCRIPTION OF THE INVENTION

Bone marrow (BM) is a highly vascular modified connective tissue that occupies the cavities of most bones. One of the well recognized functions of BM is production of new blood cells. Worn blood cells are periodically removed from the circulation and must be replaced. New blood cells arise from cells in the BM known as stem cells or stem/progenitor cells. Bone marrow has long been recognized as a rich source of many types of stem/progenitor cells, and those that give rise to blood cells have been extensively characterized. Under appropriate conditions certain stem/progenitor cells divide and differentiate along recognized pathways to form blood cells, such as those of the erythroid, myeloid, and lymphoid lineages.

Recently there has been increasing appreciation that BM from adult subjects is not restricted to production of new blood cells, but also is a source of multipotent stem/progenitor cells with potential to give rise to cells that differentiate into many other lineages, including endothelial cells (EC), muscle cells (MC) (including cardiac muscle cells of the heart (CMC)) and others.

As discussed herein, the invention relates to the production and use of genetically modified hematopoietic stem cells, such as genetically modified bone marrow cells (GMBMC) (also referred to herein as “transgenic bone marrow cells,” “transgenic BMC”; “transgenic bone marrow-derived cells” or “transgenic BMDC”) or genetically modified cells derived from cord blood. By “cord blood cells” is meant any cell derived from a human umbilical cord. Such cells are useful in a method of the invention. The transgenic cells carry transgenes that are stably integrated into the genomes of the cells. As a result of such integration, the transgenes are passed on to the progeny of these cells. Thus the progeny are also transgenic cells. As used herein, the terms “transgene,” refers to a heterologous gene, or recombinant construct of multiple genes (“gene cassette”) in a vector. A “transgenic cell” is a cell into which a vector comprising a transgene has been introduced. The terms “transduced,” “transduction,” and the related terms “transformed,” “transformation,” “gene transfer” and the like as used herein refer to process of being made transgenic, or the state of being transgenic. In some contexts, the terms can be used synonymously. For example a “transgenic cell” can also be referred to as a “transduced cell.” A transduced cell can also be a cell infected with a viral vector such as a rAAV vector. A “stably transduced” or “stably transformed” cell refers to a cell in which the transgene is stably integrated into the genome of the cell and is accordingly passed on to daughter cells by division.

As further described below, the transgenes expressed by the GMBMC or GMBMDC can include a wide array of therapeutic or reporter genes. Accordingly the transgenic cells of the invention have a wide spectrum of important uses. The transgenic cells, when introduced into a host animal can express the transgene for extended periods in vivo, and can be tracked and distinguished from native cells of the host by virtue of expression of the transgene.

The transgenic cells can be used for a variety of therapeutic purposes, including but not limited to use in the prevention, treatment or alleviation of symptoms associated with a cardiovascular disorder, particularly those coronary diseases directly or indirectly associated with ischemia (myocardial ischemia), an infarct (myocardial infarction), congestive heart failure (CHF) and related heart muscle disorders, such as cardiomyopathy and cardiomyositis. By “treat” is meant decrease, suppress, attenuate, diminish, arrest, or stabilize the development or progression of a disease.

### Stem Cells Useful in the Methods of the Invention

Hematopoietic stem cells are a broad class of cells that encompass many different cell types useful in the methods of the invention, such cells include bone marrow-derived cells and cord blood cells. As discussed, the GMBMC and GMBMDC of the invention include isolated transgenic cells derived from bone marrow. As used herein, a “cell derived from bone marrow” (whether or not transgenic) is meant to refer to any cell type that is either 1) directly obtained from the BM of an animal, or 2) the product of a cell that is directly obtained from the BM. Examples of the former type of cells, in particular various characterized stem/progenitor cells in the BM, are further described infra. A “cell derived from bone marrow” can also refer to a cell of the second type described above, for example a progeny cell that arose by division and/or differentiation of a cell type of the BM, including those that arise, for example, in a part of the body remote from the BM upon exit of a BM cell from the BM (for example, exit of the cell into the blood stream). As discussed, well known examples of such progeny cells include: red blood cells (the differentiated product of BM stem/progenitor cells of the erythroid lineage); various nucleated blood cells (granulocytes) including polymorphonuclear leukocytes, eosinophils, basophils, macrophages and monocytes (differentiated cells produced by BM cells of the myeloid lineage) and lymphocytes (differentiated cells of the BM lymphocytic lineage).

Other cells “derived from bone marrow” include any of the progeny cells that arise by division and/or differentiation of a “multipotent stem/progenitor cell” of the BM, as discussed below. Examples of such progeny cells include but are not limited to endothelial cells (EC) and muscle cells (MC), including cardiac muscle cells (CMC) of the heart. As used herein, a “cell derived from bone marrow” can include stem cell/progenitor cells present in the blood or matrix of the umbilical cord or placenta.

A “transgenic cell derived from bone marrow,” as used herein, refers to either: 1) a cell isolated from BM and transduced with an expression vector according to the invention, or 2) a transgenic cell that is the product (progeny) of said cell, whether or not the cell is located in the BM. It will be readily apparent to those of skill in the art that in some embodiments the transgenic BM-derived cells of the invention, when administered to a host subject, can divide and/or differentiate along multiple lineages, as well as migrate to many differ sites in the body.

By “isolated” as it is used herein to refer to transgenic cells and BM-derived cells in vitro, or in the form of a pharmaceutical product, is meant that the cells have been separated from bone marrow and other cell substituents that naturally accompany it. Preferably, the cells of the invention are at least 80% or 90% to 95% pure (w/w). Desirably, a transgenic cell is at least 98% to 99% homogeneity (w/w). Such cells are useful for many pharmaceutical, clinical and research applications. Once substantially purified or isolated, the transgenic cells would be substantially free of unwanted marrow contaminants. Once purified partially or to substantial purity, the transgenic cells are suited for therapeutic or other uses such as those provided herein. Purity can be determined by a variety of standard techniques such as cell culture, microscopic and centrifugation techniques (e.g., Ficoll gradient) and cell sorting such as fluorescence activated cell sorting (FACS), for example for detection and/or selection of cells bearing particular markers of cell lineage.

Human umbilical cord blood (“cord blood”) is a rich source of mesenchymal stem cells (MSCs). Methods of isolating such cells are known in the art. Briefly, a 1 ml portion of umbilical cord is placed in a well containing RPMI and 20% FBS. The matrix cells migrate out from the cord and adhere to the plastic well. Such cells have a fibroblast morphology. The supernatant and tissue are discarded after several days in culture. The cells remaining in the well are trypsinized and transferred to a secondary culture for expansion. See, for example, Connealey et al., Proc. Natl. Acad. Sci. USA, Vol. 94, pp. 9836-9841, September 1997; and Meagher and Klingemann et al., J Hematother Stem Cell Res. 2002 June; 11(3):445-8. J Hematother Stem Cell Res. 2002 June; 11(3):445-8. While particular examples are directed to bone marrow-derived cells, one skilled in the art appreciates that any hematopoietic stem cell may be used in the methods of the invention.

For transduction of maximal numbers of cells, or for transduction of multiple cellular lineages, a whole cell isolate or a culture of whole cells may be used. The art is well advanced in the areas of isolation of BM from bones of host subjects, tissue culture methods for propagation of BM cells, and of subpopulations of BM cells enriched in particular lineages, providing many options for selecting a starting population of BM cells to be transduced in accord with the invention. For example, whole BM can be cultured and subjected to gene transfer by contacting either freshly isolated whole BM, or a whole BM culture, with a vector comprising a gene of interest. Depending on the purpose, in many applications it may be preferable to use an enriched population of BM cells as the starting material.

BM cells can be isolated for example by flushing the cells from long bones such as the femur or tibia. Mononuclear cells in the BM can be isolated by gradient centrifugation and cultured, for example as described in [13, 14] and Examples below. As further shown below, transduction of BM cultures, for example with a recombinant adeno-associated viral (rAAV) vector can result in stable transduction of cells that when administered to a host subject by BM transplantation can populate the peripheral blood, spleen and cardiac tissue. The transplanted cells can integrate into the host tissues and can express the transgene for at least 1, 2, 3, 4, 5, 6, 7, or 8 (until 12 weeks) weeks after administration of the cells to the subject. In some embodiments, expression can continue for three, six, nine or even twelve months following administration to a subject.

For some applications it is desirable to select and enrich a particular cell type of the BM before expansion in vitro and transfection. One useful method for obtaining a selected population of isolated BM cells involves clonal expansion, which can include at least one of and preferably all of the following process steps:
a) collecting BM cells from a mammal which cells have a size of less than about 100 microns, preferably less than about 50 microns, more preferably about 40 microns or less,
b) culturing (expanding) the collected cells in medium under conditions that select for adherent cells,
c) selecting the adherent cells and expanding those cells in medium to semi-confluency,
d) serially diluting the cultured cells into chambers with conditioned medium, the dilution being sufficient to produce a density of less than about 1 cell per chamber to make clonal isolates of the expanded cells; and
e) culturing (expanding) each of the clonal isolates and selecting chambers having expanded cells to make the population of isolated BM cells. If desired, the cells can be treated with a tyrosine kinase inhibitor, e.g., genistein, prior to infection with a rAAV.

More specifically, and in embodiments in which human BM cells are desired, (for example hBMSC multipotent stem cells, described infra) BM cells can be obtained by taking fresh unprocessed BM cells from young male donors. Alternatively, such cells can be purchased. The cells are typically separated from blood cells by centrifugation, hemolysis and related standard procedures. The BMs are washed in an acceptable buffer such as DPBS and filtered to collect cells having a size less that about 100 microns, preferably less than about 50 microns, more preferably about 40 microns. A standard nylon filter, for instance, can be used.

Once isolated, the BMs are grown in a complete culture medium with low glucose (e.g., DMEM) that contains a rich source of growth factors and cytokines. Fetal bovine serum (FBS) is preferred. Cells are cultured (i.e., expanded) for less than about two weeks, preferably about a week or less such as four to six days. The conditioned medium is then replaced with fresh medium, adherent cells are removed from the culture dishes and resuspended in fresh medium to select cells that can be expanded. The selected cells are grown to semi-confluency (between 50% to 90% confluent) and again, adherent cells are selected. Such cells are then reseeded in complete medium in a tissue culture flask at a density of about 10⁴ cells per centimeter.

After the cells reach semi-confluency, they are reseeded (serially) into the flasks at the same or similar density. The cultures are preferably passaged more than one time, typically less than five times and preferably about two times to continue selection for expanding cells. Selected cells are then serially diluted into single well chambers (e.g., standard 96-well plate) at a density of less than about 1 cell per chamber, preferably ½ a cell per chamber. Preferably, the cells are cultured with conditioned media to promote growth to sub-confluence (i.e., less than 50% confluent). Wells with expanded cell clones are expanded and replated as needed. Depending upon the type of cell desired for transduction, suitable protocols and culture conditions can be used to culture and expand populations comprising a single desired cell type.

Certain transgenic cells derived from BM in accord with the invention are isolated stem or progenitor cells of the BM. In general, “stem cells” refer to unspecialized human or animal cells that can produce mature specialized body cells and at the same time replicate themselves. Embryonic stem cells are derived from a blastocyst, which is a very early embryo that contains 200 to 250 cells and is shaped like a hollow sphere. The embryonic stem cells are cells in the blastocyst that ultimately would develop into a person or animal.

By contrast, “adult” stem cells are derived from the umbilical cord and placenta or from blood, bone marrow, skin, and other tissues. The similar embryonic germ line cells come from a fetus that is 5 to 9 weeks old and are derived from tissue that would have developed into the ovaries or testes. “Progenitor cells” are stem cells that are committed to differentiate along a more restricted lineage, for example to form heart muscle, nervous tissue, etc.

The invention takes advantage of the rich population of naturally occurring stem or progenitor cells of the BM, which include but are not limited to the following recognized cell types: endothelial progenitor cells (EPC); hematopoietic stem cells (HSC); mesenchymal stem cells (MSC); multipotent adult progenitor cells (MAPC) as described by Jiang et al. [17] and human multipotent BM stem cells (hBMSC), the latter as described in copending PCT application US04/63298, incorporated by reference herein in its entirety.

More specifically, “hBMSC” are isolated multipotent BM stem cells that exhibit reduced levels of at least one of the following markers: CD90, CD117, CD34, CD113, FLK-1, tie-2, Oct 4, GATA-4, NKx2.5, Rex-1, CD105, CD117, CD133, MHC class I receptor and MHC class II receptor, as determined by a standard cell marker detection assay. In one embodiment, levels of one or more (1, 2, 3, 4, 5, 6, 7, 8, 9, 10, 11, 12, 13, 14, or 15) markers are reduced to such a degree as to be undetectable use a standard assay (e.g., Western blot, immunocytochemistry).

Further information regarding making and using such particular stem cells can be found, for instance, in U.S. Provisional Application No. 60/515,320 as filed on Oct. 28, 2003. See also PCT/US2004/36298 as filed on Oct. 28, 2004. The disclosures of the 60/515,320 and PCT/US2004/36298 applications are each incorporated by reference.

By the phrase “standard cell marker detection assay” is meant a conventional immunological or molecular assay formatted to detect and optionally quantitate one or more of the cell markers described herein (e.g., CD34, CD90, CD117, etc.). Examples of such conventional immunological assays include Western blotting, ELISA, RIA and fluorescence activated cell sorting (FACS). FACS is an automated or semi-automated method that is preferred for embodiments needing larger amounts of transduced cells.

Preferred antibodies for use in such assays are provided below. See generally, Harlow and Lane in Antibodies: A Laboratory Manual, CSH Publications, N.Y. (1988), for disclosure relating to these and other suitable assays. Particular molecular assays suitable for such use include polymerase chain reaction (PCR) type assays using oligonucleotide primers, for instance. See WO 92/07075 for general disclosure relating to recombinant PCR and related methods. See also Sambrook et al. in Molecular Cloning: A Laboratory Manual (2d ed. 1989); and Ausubel et al., Current Protocols in Molecular Biology, John Wiley & Sons, New York, 1989; for general disclosure relating to recognized immunological and molecular assays that can be used to detect cell markers.

Certain BM-derived transgenic stem or progenitor cells in accord with the invention are endothelial progenitor cells (EPC). EPC are capable of forming endothelial cells (ECs), for instance, after contact with EC promoting conditions as determined by a standard EC differentiation assay. Examples of such EC promoting conditions are known in the field and include contact with certain angiogenic factors and cell mitogens such as those disclosed by U.S. Pat. No. 5,980,887; PCT/US99/05130 (WO 99/45775) and references cited therein. Angiogenic factors and mitogens include acidic and basic fibroblast growth factors (aFGF and bFGF), vascular endothelial growth factor (VEGF-1), VEGF165, epidermal growth factor (EGF), transforming growth factor α and β (TGF-α and TFG-β), platelet-derived endothelial growth factor (PD-ECGF), platelet-derived growth factor (PDGF), tumor necrosis factor cc (TNF-α), hepatocyte growth factor (HGF), insulin like growth factor (IGF), erythropoietin, colony stimulating factor (CSF), macrophage-CSF (M-CSF), granulocyte/macrophage CSF (GM-CSF), angiopoetin-1 (Ang1) and nitric oxidesynthase (NOS); and functional fragments thereof. Muteins or functional fragments of a mitogen may be used as long as they induce or promote formation of ECs.

A “functional fragment” is a portion of a polypeptide or nucleic acid molecule that is of a length sufficient to have at least one biological activity attributed to the polypeptide or nucleic acid molecule from which the fragment is derived. Exemplary biological activities of a therapeutic polypeptide include reducing apoptosis, increasing angiogenesis, or increasing proliferation of a cell of interest.

Assays for measuring cell apoptosis are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

Methods for measuring an increase in angiogenesis are also known in the art and are described herein. In general, angiogenesis can be assayed by measuring the number of non-branching blood vessel segments (number of segments per unit area), the functional vascular density (total length of perfused blood vessel per unit area), the vessel diameter, or the vessel volume density (total of calculated blood vessel volume based on length and diameter of each segment per unit area). Angiogenesis can also be quantitated using endothelial cell markers. For example, angiogenesis can be assayed in a cardiac tissue using the following method. Twenty-two weeks after myocardial infarction, tissue samples were fixed with 4% paraformaldehyde, and immunohistochemical staining was performed using antibodies prepared against a rat specific endothelial cell marker isolectin B4 (Vector Laboratories). Capillary density was evaluated morphometrically by histological examination of five randomly selected fields of tissue sections of peri-infarct left ventricular myocardium. Capillaries were recognized as tubular structures positive for isolectin. Such methods are described, for example, by Iwakura et al., Circulation 2003; 108: 3115-21.

Methods of assaying cell growth and proliferation are known in the art. See, for example, Kittler et al. (Nature. 432 (7020):1036-40, 2004) and Miyamoto et al. (Nature 416(6883):865-9, 2002). Assays for cell proliferation generally involve the measurement of DNA synthesis during cell replication. In one embodiment, DNA synthesis is detected using labeled DNA precursors, such as ([³H]-Thymidine or 5-bromo-2*-deoxyuridine [BrdU], which are added to cells (or animals) and then the incorporation of these precursors into genomic DNA during the S phase of the cell cycle (replication) is detected (Ruefli-Brasse et al., Science 302(5650):1581-4, 2003; Gu et al., Science 302 (5644):445-9, 2003).

Assays for measuring cell survival are known in the art, and are described, for example, by Crouch et al. (J. Immunol. Meth. 160, 81-8); Kangas et al. (Med. Biol. 62, 33843, 1984); Lundin et al., (Meth. Enzymol. 133, 2742, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

A preferred EC promoting condition includes contact with VEGF, particularly VEGF-1, VEGF165, or both. Additionally preferred EC promoting conditions include contact with certain cell matrix proteins, such as fibronectin.

By the phrase “standard EC differentiation assay” is meant any of the assays used to detect and monitor function of ECs, such as those disclosed by U.S. Pat. No. 5,980,887 and WO 99/45775. A preferred assay involves detection of EC specific markers, such as UEA-1, CD34, CD31, Flk-1, Tie-2 and E-selectin.

Also preferred are transduced BM stem or progenitor cells that can form smooth muscle cells (SMC), particularly after contact with SMC promoting conditions as determined by a standard SMC differentiation assay. Typical SMC promoting conditions are known and include contact with at least one angiogenic factor or cell mitogen. A preferred SMC promoting condition involves contact with a platelet derived growth factor (PDGF) including muteins or active fragments thereof.

By the phrase “standard SMC differentiation assay” is meant an immunological or molecular test (e.g., ELISA, Western blot, FACS analysis or PCR) that is capable of detecting and optionally quantitating at least one and preferably all of the following SMC specific markers: αSMA, PDGFβ receptor, SM22α, and SMI.

### Stably Transduced Cells

The transgenic cells of the invention are stably transduced with an expression vector comprising a nucleic acid sequence encoding a therapeutic gene or a reporter gene of interest. This transduction can be carried out in vitro or in vivo. Preferably the cells are stably transduced with a viral vector (e.g., rAAV). In one embodiment, the BM cells of a donor subject are genetically modified by contacting a population of isolated BM cells from the subject with an expression vector in vitro.

As discussed, the invention has a very broad range of uses. The selection of the gene of interest (either therapeutic gene or reporter gene) for stable integration into the cells will accordingly be guided by the intended purpose.

For certain applications of the inventive methods and compositions, a preferred therapeutic gene of interest is a trophic hormone, expressed as a transgene from a GMBMC of the invention (preferably from a rAAV vector). There is general recognition that stimulation of tissues with trophic hormones may improve diverse organ-specific processes of aging and atrophy. As an example, one preferred protein, human growth hormone (hGH), is a candidate for gene therapy for dilated cardiomyopathy, based on clinical and animal studies indicating that long-term administration of hGH protein may be beneficially for weakened cardiomyocytes [36-38]. Local production of therapeutic secretable proteins by the rAAV-bearing cells of the invention may provide the advantages of higher concentrations in the target organ and fewer systemic side effects. For some applications greater therapeutic benefits are attainable via local production than by systemic administration of hGH protein, or local gene therapy using vectors delivered directly to the affected tissues.

Other preferred therapeutic genes of interest include angiogenic proteins. As used herein, an “angiogenic protein” refers to any protein or fragment thereof that promotes formation of blood vessels (i.e., promotes angiogenesis or vasculogenesis). Such disclosed factors and mitogens include acidic and basic fibroblast growth factors (aFGF and bFGF), vascular endothelial growth factor (VEGF-1), VEGF165, epidermal growth factor (EGF), transforming growth factor α and β (TGF-α and TFG-β), platelet-derived endothelial growth factor (PD-ECGF), platelet-derived growth factor (PDGF), tumor necrosis factor α (TNF-α), hepatocyte growth factor (HGF), insulin like growth factor (IGF-1, IGF-2), erythropoietin, colony stimulating factor (CSF), macrophage-CSF (M-CSF), granulocyte/macrophage CSF (GM-CSF), angiopoetin-1 (Ang1) and nitric oxide synthase (NOS), and functional fragments thereof.

A particularly preferred angiogenic factor for therapeutic purposes in cardiovascular applications is insulin-like growth factor-1 (IGF-1). This factor is recognized as a survival growth factor for the heart. Heart failure is a significant cause of morbidity and mortality, with a current US prevalence of 5 million and 5-year survival near 50%. Systemic administration with insulin-like growth factor-1 (IGF-1) attenuates ischemia-induced myocardial injury (cell damage due to low blood flow and hypoxia) in mice. In addition, recently, IGF-1 expression by rAAV was shown to improve skeletal muscle atrophy in mouse models of amyotrophic lateral sclerosis (ALS), dramatically prolonging the life of these animals. Amyotrophic lateral sclerosis (ALS) is progressive, lethal neuromuscular disease that is associated with the degeneration of spinal and brainstem motor neurons, leading to atrophy of limb, axial and respiratory muscles.

As shown herein, overexpression of IGF-1 by rAAV vectors can be achieved in vitro and is predicted to become a protective therapy for cardiomyocytes experiencing myocardial ischemia in vivo. See, for instance, Examples 3-7 infra. Such a therapy could improve both acute and chronic forms of cardiac dysfunction and heart failure. The protective gene could be delivered to cells of the heart by appropriate selection of a starting population of BM cells, and stable integration of the IGF-1 transgene into the cells, to provide lines of transgenic cells useful for such an approach. Upon administration to a subject in need, the cells could integrate into cardiac tissue and provide a local source of IGF-1, sufficient inter alia to promote cardiac cell proliferation and to reduce apoptosis.

Yet other preferred therapeutic genes of interest, depending upon the intended use for the transgenic cells, are hematopoietic proteins. A “hematopoietic protein” is defined as any protein or functional fragment thereof that stimulates development, differentiation and/or proliferation of blood cell precursors. Preferred hematopoietic proteins include granulocyte-macrophage colony-stimulating factor (GM-CSF), VEGF, Steel factor (SLF, also known as Stem cell factor (SCF)), stromal cell-derived factor (SDF-1), granulocyte-colony stimulating factor (G-CSF), HGF, angiopoietin-1, angiopoietin-2, M-CSF, b-FGF, and FLT-3 ligand.

By the term “vector” is meant a recombinant plasmid or viral construct used as a vehicle to introduce one or more transgenes into a cell. Preferred vectors for in vivo use in subjects are viral vectors, and as discussed, particularly preferred viral vectors are rAAV vectors. As used herein, vector is a term referring to a sequence of genetic material into which a nucleotide sequence (or “transgene”, typically a fragment of DNA encoding a polypeptide of interest) has been inserted and which can be used to introduce exogenous genetic material into a cell or into the genome of an organism. An “expression vector” is vector used to introduce a DNA or RNA sequence into a cell, causing the product of the DNA or RNA (typically a protein or polypeptide) to be produced by the cell.

Typically a mammalian expression vector utilizes a promoter adjacent to a transgene to express the corresponding mRNA that can be translated to the corresponding protein or polypeptide in the cell. As used herein, a “promoter” refers to a DNA sequence to which RNA polymerase binds to initiate transcription of messenger RNA, and to which other regulatory elements bind to facilitate, regulate, enhance or suppress transcription. A promoter that is “operably linked” to a DNA sequence encoding a gene or a fragment thereof in a vector causes the DNA sequence to be expressed or produced when the vector is introduced into a cell or is provided with suitable substrates and conditions in vitro. The promoter of the invention can be a “ubiquitous” promoter active in essentially all cells of a host organism (such as a human), for example, a CMV, beta-actin or optomegalovirus promoters, or it may be a promoter whose expression is more or less specific to the target cell or tissue, or to the oncogene. An example of a useful promoter which could be used to express a gene of interest according to the invention is a cytomegalovirus (CMV) immediate early promoter (CMV IE) (Xu et al., Gene 272: 149-156, 2001). These promoters confer high levels of expression in most animal tissues, and are generally not dependent on the particular encoded proteins to be expressed. Examples of other such promoters of use in the invention include Rous sarcoma virus promoter, adenovirus major late promoter (MLP), Herpes Simplex Virus promoter, HIV long terminal repeat (LTR) promoter, beta actin promoter (Genbank Accession No. K00790), or murine metallothionein promoter (Stratagene San Diego Calif.). Examples of tissue- or cell-specific promoters are described infra. The latter type of promoters can be used to advantage, for example to restrict expression of transgenes to cells having tropism for particular serotypes of rAAV.

As discussed, transfection refers to a process of delivering heterologous DNA, such as a viral vector encoding a transgene of interest, or plasmid DNA to a cell by physical or chemical methods. The DNA is transferred into the cell by any suitable means, such as electroporation, calcium phosphate precipitation, or other methods well known in the art. Use of the term “transduction” encompasses both introducing the gene or gene cassette into a cell for purposes of tracking (as with a reporter gene), or for delivering a therapeutic gene or correcting a gene defect in a cell. Transduction in the context of producing viral vectors for gene therapy (for example rAAV vectors) in a cell can also mean introduction of a gene or gene cassette into a producer cell to enable the cell to produce rAAV. The rAAV particles made by the producer cells are subsequently purified by standard methods known in the art and as described below.

As discussed above, typical transgenes comprise a heterologous gene sequence, or a recombinant construct of multiple genes (“gene cassette”) in a vector. The recombinant AAV vectors of the invention can be produced in vitro by introducing gene constructs into cells known as producer cells. The term “producer cell” refers one of many known cell lines useful for production of rAAV, into which heterologous genes are typically introduced by viral infection or transfection with plasmid DNA. As used herein, the term “infection” refers to delivery of heterologous DNA into a cell by a virus. Infection of a producer cell with two (or more) viruses at different times is referred to as “co-infection.”

In general, systems for producing rAAV comprise three basic elements: 1) a gene cassette containing one or more genes of interest, 2) a gene cassette containing AAV rep and cap genes and 3) a source of “helper” virus proteins.

Typically the first gene cassette is constructed with the gene of interest flanked by inverted terminal repeats (ITRs) from AAV. Particular genes of interest of use in the invention have been described supra. A suitable vector for expressing one or more reporter genes is pAAV-CMV-lacZ [39]. This vector comprises a CMV promoter and drives expression of the lacZ gene. For more restricted expression of transgenes, other suitable vectors are constructed with cell-specific promoters, such as the vector described in [9] which restricts expression of the transgene to cardiac muscle cells. Other suitable promoters are described infra.

As discussed, preferred transgenic cells of the invention are stably transduced with the rAAV vectors. Within the rAAV system, ITRs function to direct integration of the gene of interest into the host cell genome, thereby facilitating stable transduction (Hermonat and Muzyczka, Proc Natl Acad Sci U S A. 81(20):6466-70, 1984; Samulski, et al., Cell. 33(1):135-43. 1983).

The second gene cassette contains rep and cap, AAV genes encoding proteins needed for replication and packaging of rAAV. The rep gene encodes four proteins (Rep 78, 68, 52 and 40) required for DNA replication. The cap genes encode three structural proteins (VP1, VP2, and VP3) that make up the virus capsid.

The third element is required because AAV-2 does not replicate on its own. “Helper functions” are protein products from helper DNA viruses that create a cellular environment conducive to efficient replication and packaging of rAAV. Adenovirus (Ad) has been used extensively to provide helper functions for rAAV. The gene products provided by Ad are encoded by the genes E1a, E1b, E2a, E4orf6, and Va (Hauswirth et al., Methods Enzymol. 316:743-61, 2000).

The rAAV vectors used to transfect the BM cells can be produced in vitro, using suitable producer cell lines, such as 293 and HeLa. Alternatively in some instances the rAAV vectors can be purchased from commercial sources. A well-known strategy for delivering all of the required elements for rAAV production utilizes two plasmids and a helper virus. This method relies on transfection of the producer cells with plasmids containing gene cassettes encoding the necessary gene products, as well as infection of the cells with Ad to provide the helper functions. This system employs plasmids with two different gene cassettes. The first is a proviral plasmid encoding the recombinant DNA to be packaged as rAAV. The second is a plasmid encoding the rep and cap genes. To introduce these various elements into the cells, the cells are infected with Ad as well as transfected with the two plasmids. Alternatively, in more recent protocols, the Ad infection step can be replaced by transfection with an adenovirus “helper plasmid” containing the VA, E2A and E4 genes (Xiao, et al., J. Virol. 72(3):2224-32. 1998, Matsushita, et al., Gene Ther. 5(7):938-45.1998).

While Ad has been used conventionally as the helper virus for rAAV production, other DNA viruses, such as Herpes simplex virus type 1 (HSV-1) can be used as well. The minimal set of HSV-1 genes required for AAV-2 replication and packaging has been identified, and includes the early genes UL5, UL8, UL52 and UL29 (Muzyczka and Burns, supra). These genes encode components of the HSV-1 core replication machinery, i.e., the helicase, primase, primase accessory proteins, and the single-stranded DNA binding protein (Knipe, Adv Virus Res. 37:85-123, 1989; Weller, J Gen Virol. 71 (Pt 12):2941-52 1991). This rAAV helper property of HSV-1 has been utilized in the design and construction of a recombinant Herpes virus vector capable of providing helper virus gene products needed for rAAV production (Conway et al., Gene Ther. 6(6):986-93, 1999).

A preferred method for preparing the rAAV vectors of the invention is described, for example, in [24]. Briefly, subconfluent 293 cells are co-transfected with vector plasmid and pLTAAV help using calcium phosphate [39]. Cells are then infected with adenovirus Ad5dl312 (an E1A-deletion mutant) at a multiplicity of infection of about 2. The E1A-deleted rAd-lacZ vector can be prepared for example as described in [40]. After approximately 72 hours, the cells are harvested and lysed by repeated (for example, three) freeze/thaw cycles. Ad is heat-inactivated, and the rAAV virions are purified, for example on cesium chloride gradients. The gradient fractions containing rAAV are dialyzed against sterile PBS, and stored at about −80° C. Particle titers (preferably of about 1˜2×10¹²/ml) can be determined, for example, by dot blot analysis.

Recombinant AAV vectors have generally been based on AAV-2 capsids. It has recently been shown that rAAV vectors based on capsids from AAV-1, AAV-3, or AAV-4 serotypes differ substantially from AAV-2 in their tropism. Capsids from other AAV serotypes offer advantages in certain in vivo applications over rAAV vectors based on the AAV-2 capsid. For example, rAAV vectors with particular serotypes may increase the efficiency of gene delivery and integration into the genome of certain types of BM stem or progenitor cells. Although it is shown in Examples below that rAAV-2 is an effective vector serotype for transduction and stable integration into BMC and BMDC, the invention is not so limited. Further, it may be advantageous to have available alternative transgenic BM cell lines comprising rAAV vectors based on multiple AAV serotypes. For example, this could become important if re-administration of BM cells comprising a rAAV vector becomes clinically necessary, because it has been demonstrated that re-administration of the same rAAV vector with the same capsid can be ineffective, possibly due to the generation of neutralizing antibodies generated to the vector (Xiao, et al., supra, Halbert, et al., J. Virol. 71(8):5932-41, 1997; Halbert, et al., Proc Natl Acad Sci USA. 94(4):1426-31, 1997). This problem can be avoided by administering a rAAV particle whose capsid is composed of proteins from a different AAV serotype, not affected by the presence of a neutralizing antibody to the first rAAV vector (Xiao, et al., supra). For the above reasons, recombinant AAV vectors constructed using cap genes from serotypes other than, or in addition to AAV-2, are desirable.

As discussed, gene therapy represents a promising approach for the treatment of many diseases, including inherited heart diseases, cardiomyopathies, and congestive heart failure. See, for example, Nabel E. G (1995) Circulation 91:541-548. Previous work has demonstrated that recombinant adenoviral (rAd) vectors can efficiently transduce cardiomyocytes in vivo to express genes, including the potassium channel, sarcoplasmic calcium ATPase-2A, and phospholamban. rAd-mediated gene transfer can be limited by immune responses to viral proteins, which can cause significant myocardial inflammation. Designing a delivery system with low cytotoxicity and cardiac-specific gene expression has been a central goal of cardiac gene therapy.

As discussed above, a preferred viral gene delivery system with low cytotoxicity is provided by vectors derived from a non-pathogenic human parvovirus [22], i.e., recombinant adeno-associated viral (rAAV). The small size and physical stability of these vectors can be advantageous for in vivo use. Transgene expression from rAAV vectors can persist in a wide range of tissues [23-25]. Moreover, there is no evidence of cell damage from inflammation after rAAV administration to the liver, skeletal muscle, brain, and heart [24, 26-28]. Accordingly, rAAV vectors have been recognized as suitable vectors for systemic and local long-term delivery of gene therapy for clinical diseases [29, 30].

In some circumstances the promiscuous tropism of rAAV may lead to the undesirable expression of therapeutic genes in non-targeted cells. This limitation may be overcome by the use of tissue-specific promoters. Liver-, brain-, cancer-, and rod photoreceptor-specific expression can be achieved, for example, using tissue-specific promoters, such as those from albumin, enolase, calcitonin, and rodhopsin, respectively [31-34]. Muscle-specific expression in skeletal muscle can be directed, for example, by a rAAV vector comprising a muscle creatine kinase (MCK) promoter [35].

For cardiac-specific expression, a suitable promoter is an alpha myosin heavy chain (MHC) gene promoter. rAAV vectors expressing a therapeutic or reporter gene under the control of a cardiac-specific promoter can be made, for example, as described in [9] by cloning fragments of the α-MHC promoter (−344 to +19), a larger promoter fragment containing the PNR (−344 to +119), or the α-MHC enhancer (−344 to −156) together with a heterologous promoter to control transgene expression. Long-term cardiac expression of both therapeutic and reporter genes with low cytotoxicity can be attained using these constructs [9].

As discussed, the vectors include a nucleic acid sequence encoding a therapeutic or reporter gene and other elements as described above, appropriate for transducing hematopoietic stem cells (e.g., bone marrow cells, cord blood cells) and achieving stable gene expression. By contacting the cells with the vector and selecting for those cells in which the vector has been stably integrated into the genome of the cell, one can obtain a virtually unlimited supply of hematopoietic stem cells (e.g., bone marrow cells, cord blood cells).

### Methods of Use

In one aspect, the invention provides methods for expressing a therapeutic or reporter gene in a cell or tissue of interest in a host subject. The methods involve the use of genetically modified (transgenic) hematopoietic stem cells (e.g., bone marrow cells, cord blood cells), such as BM-derived cells from a donor subject, prepared as described above. In one embodiment, BM cells are obtained from the donor subject and following genetic modification, the cells are administered to a recipient (host) subject, for example by transplantation of a cell population or a graft of transgenic cells.

A “donor” is defined as the source of the transgenic BMC or BMDC, whereas a “recipient” or “host” is the subject that receives the graft. Immunological relationship between the donor and recipient can be allogenic, autologous, or xenogeneic as needed. In preferred invention embodiments, the donor and recipient will be genetically identical and usually will be the same individual (syngeneic). In this instance, the graft will be syngeneic with respect to the donor and recipient. In the case of syngeneic transplantation, the BM cells are manipulated ex vivo (typically including in vitro expansion of the cell numbers before and/or after gene transfer) and then reintroduced into the donor subject.

The term “graft,” as used herein, includes (or in some embodiments consists of) the isolated transgenic hematopoietic stem cells or BM-derived cells described herein. A “graft” can also refer to a cell or tissue preparation that includes the GMBMC or GMBMDC and optionally other cells, such as ECs, SMCs and CMCs from a mammal.

By “graft” is also meant hematopoietic stem cells, GMBMC or GMBMDC of the invention, which have been administered to a recipient and become part of one or more tissues or organs of that recipient. Sometimes the word “engraftment” will be used to denote intended assimilation (incorporation) of the transgenic cells into a targeted tissue or organ (either as BMC or differentiated cells). Preferred engraftments involve assimilation into tissues, such as blood, spleen and cardiovascular tissue. A particularly preferred engraftment involves assimilation of the cells into cardiovascular tissues (particularly blood vessels, such as veins, arteries, etc.) and into cardiac tissue, for example into cardiac muscle.

A graft of the invention may also take the form of a tissue culture preparation in which the transgenic cells of the invention have been combined with other cells and/or mitogens to promote differentiation and/or cell replication that produces an intended graft. If desired, the preparation can be combined with synthetic or semi-synthetic fibers to give structure to the graft. Fibers, such as Dacron, Teflon or Gore-Tex are preferred for certain applications.

A particular example of a graft of the invention is a preparation of transgenic hBMSCs that have been prepared from BM of a donor and genetically modified to prevent, treat or reduce the severity of a cardiovascular disorder, such as myocardial ischemia or an infarct. The preparation can include a pharmaceutically acceptable carrier, such as saline and optionally may include at least one of a mitogen, an angiogenic factor, and a cell type, such as a CMC, EC, EPC, or SMC to assist an intended engraftment result.

The transgenic cells of the invention can be administered to a host subject by any medically acceptable means. In certain preferred applications, the cells are administered to the BM of the subject. In other applications, the cells are administered to the bloodstream of the subject. In yet other embodiments, the cells are administered to the heart or a cardiovascular tissue of a subject.

It is an object of the present invention to provide transduced cells (e.g., GMBMCs) to prevent, treat or reduce the severity of a cardiac disease or disorder, such as cardiac ischemia. For example, a cardiac ischemia resulting from or associated with a myocardial infarct. In one embodiment, such methods include the steps of isolating suitable BM cells from a subject (e.g., a mammal, preferably a human patient) and transducing the cells ex vivo as described herein. Such cells include but are not limited to the particular multipotent and bone marrow derived stem cells described in the PCT/US2004/36298 application. Alternatively, nearly any of the other procedures for isolating cells disclosed herein can be used. Typical amounts of transduced cells to use will depend recognized parameters including the cardiac disease to be treated, and in the case of ischemia, the severity of the infarct. For most applications between from about 10³ to about 10⁷ transduced cells will suffice, typically about 10⁵ of such cells. Cells may be administered by any acceptable route, including suspending the cells in saline and administering the cells to a tissue of a subject with a needle, stent, catheter or similar device. In embodiments in which myocardial ischemia or an infarct is to be addressed, the administration will be a bolus injection near or directly into the site of injury. In other embodiments less invasive methods may be indicated, such as intravenous injection of hematopoietic stem cells, such as GMBMCs, for instance. The foregoing administration protocols will be generally suitable for most therapeutic methods disclosed herein.

In another embodiment, the foregoing method further includes administering to the mammal in need of treatment (e.g., a human patient) at least one angiogenic factor or mitogen (or functional fragment of the factor or mitogen). Preferred angiogenic factors and mitogens (and methods of use) are disclosed herein as well as U.S. Pat. No. 5,980,887 and WO 99/45775. Alternatively, or in addition, the method can include administering to the mammal at least one nucleic acid encoding at least one angiogenic factor or functional fragment thereof. Methods for administering such nucleic acids to the mammal have been disclosed by U.S. Pat. No. 5,980,887 and WO 99/45775, for instance. Treatment with the angiogenic factor/mitogen protein or nucleic acid encoding these can precede use of the transduced hematopoietic stem cells (e.g., bone marrow cells, cord blood cells) or it can be used during or after such treatment as needed. A particular angiogenic factor of interest is vascular endothelial growth factor (VEGF).

In yet another embodiment, the foregoing method further includes administering to the mammal endothelial progenitor cells (EPCs). This invention embodiment especially finds use where good vascular growth is needed to address a cardiovascular disorder. Methods for making and using EPCs have been disclosed. See U.S. Pat. No. 5,980,887, for example. Typical methods can include isolating the EPCs from the mammal and contacting the EPCs with at least one angiogenic factor and/or mitogen ex vivo.

The invention is broadly applicable to the prevention and treatment of a wide variety of cardiovascular disorders including congestive heart failure (CHF), ischemic cardiomyopathy, myocardial ischemia, and an infarct. If desired, such methods can further include monitoring cardiac function in the mammal seeking treatment, e.g., by monitoring at least one of echocardiography, ventricular end-diastolic dimension (LVEDD), end-systolic dimension (LVESD), fractional shortening (FS), wall motion score index (WMSI) and LV systolic pressure (LVSP). Preferred invention methods involving prevention or treatment of a particular cardiovascular disease will manifest good cardiac function as exemplified by one or more of these tests. By “good” is meant at least a 10% improvement, preferably at least 20% or 30% compared to a control that does not receive an invention composition (or receives a placebo). Particular methods for performing these tests are known in the field. A “therapeutically effective” treatment method in accord with the invention is one that provides for at least a 10% improvement in cardiac function, for a reduction in apoptosis, for an increase in angiogenesis, or for any other improvement in the health or function of a cardiac tissue. Alternatively, the improvement is by at least 25%, 50%, 75%, or 100%.

### EXAMPLES

The invention is further illustrated by reference to the following non-limiting examples.

### Example 1

### Materials and Methods

The following materials and methods were used as needed to conduct studies outlined in the Examples below.

1. Cell Culture and rAAV Vector Production and Infection.

BM cells were isolated and cultured in phenol red-free EC basal medium EBM-2 (Clonetics) supplemented with 5% fetal bovine serum (FBS), antibiotics and growth factors (EPC medium) on surfaces coated with rat plasma vitronectin (Sigma) in 0.5% gelatin solution.

In some cultures enriched for endothelial precursor cells (EPC), four days after culture, EPC (recognized as attaching spindle-shaped cells) were assayed by co-staining with detectably labeled acetylated LDL (acLDL-DiI; Biomedical Technologies) and BS-1 lectin, conjugated with FITC (Sigma). Labeling with these probes is an identifying characteristic of the endothelial lineage. Fluorescence microscopy identified double-positive cells as EPC. In some studies, the cells were analyzed by FACS for expression of Sca-1-FITC (Pharminogen) and Flk-1 (Santa Cruz) conjugated to a phycoerythrin-labeled secondary antibody (Sigma) as described [21].

Vectors (rAAV-CMV-lacZ) were prepared as described [9]. The rAAV vectors had a particle titer of 5×10¹¹ to 2×10¹²/ml. Bone marrow cells (5×10⁶ cells) were grown on 12-well plates and infected with 1000 particles/cell of rAAV-CMV-lacZ and cultured in phenol red-free EC basal medium (Clonetics, San Diego, Calif.) medium supplemented with 5% fetal bovine serum, antibiotics, and growth factors in an incubator at 37° C., as previously described [13, 14]. After addition of the vectors the culture dishes were shaken every 10 minutes for the first 30 minutes and then incubated for another 1.5 hours. Two hours after infection of the cells with the viral particles, the infected cells were used for transplantation into irradiated mice as described below.

### 2. Animal Studies

All procedures were performed in accordance with St. Elizabeth's Institutional Animal Care and Use Committee. Male FVB wild-type mice (Jackson Laboratory, Bar Harbor, Me.) were used, and bone marrow transplant (BMT) mice were created by transplantation of wild type BM with or without rAAV infection, or BM of Rosa mice. Recipient mice were lethally irradiated with 9.0 Gy, and BMT from each mouse was performed.

To examine the effect of VEGF on BM cell mobilization, we injected the mice with 100 ng VEGF-C intaperitoneally for 3 days.

To produce myocardial infarction, mice were anesthetized and intubated and stenosis was induced by ligating the left anterior descending coronary artery with 8-0 prolene suture.

For detection of β-galactosidase protein, freshly excised tissues in placed in O.C.T. compound (Sakura), flash frozen and sectioned. After fixation the slides were stained overnight with 5-bromo-4-chloro-3-iodolyl-beta-D-galactopyranoside (X-gal) as described [9].

### 3. Fluorescence-Activated Cell Sorting (FACS) Analysis

Freshly isolated BM cells or PB cells were subjected to FluoReporter lacZ Flow Cytometry Kit (Molecular Probes, OR) or stained with acetylated low-density lipoprotein (LDL) labeled with 1′-dioctadecyl1-1,3,3,3′-tetramethyl-indocarbocyanine; Di-I (Biomedical Technologies Inc, MA) as described previously [13]. Analysis was performed on a FACStar flow cytometer (Becton Dickinson) and Cell Quest Software counting 10,000 events per sample.

### 4. PCR Detection of Viral Genome

Total DNA was extracted from tissues using Puregene DNA Isolation Kit (Flowgen Bioscience, Wilford UK), and then a 286 base pair fragment of the rAAV genome was amplified using sense primer 5′-CAACTCCATCACTAGGGGT-3′(SEQ ID NO:1) of the ITR region and antisense primer 5′-AAAGTCCCTATTGGCGTTA-3′ (SEQ ID NO:2) of the CMV promoter.

### 5. Statistical Analysis

All values are expressed as mean±S.E. Statistical significance was evaluated by means of the Wilcoxon rank-sum test for comparisons between two groups. A value of P<0.05 was considered statistically significant.

### Example 2

### Transduction of BM Cells with rAAV

To test the capacity of rAAV vectors to transduce BM cells in vivo, we examined whether rAAV could be used to stably transduce BM cells by bone marrow transplantation (BMT). BM cells from donor FVB mice were infected with 1000 particles per cell of rAAV-CMV-lacZ vectors for 2 h. The infected cells were then injected into lethally irradiated recipient mice. To detect lacZ expression in the rAAV-infected cells, we determined the presence of lacZ positive cells using FACS analysis with an anti-β-gal antibody.

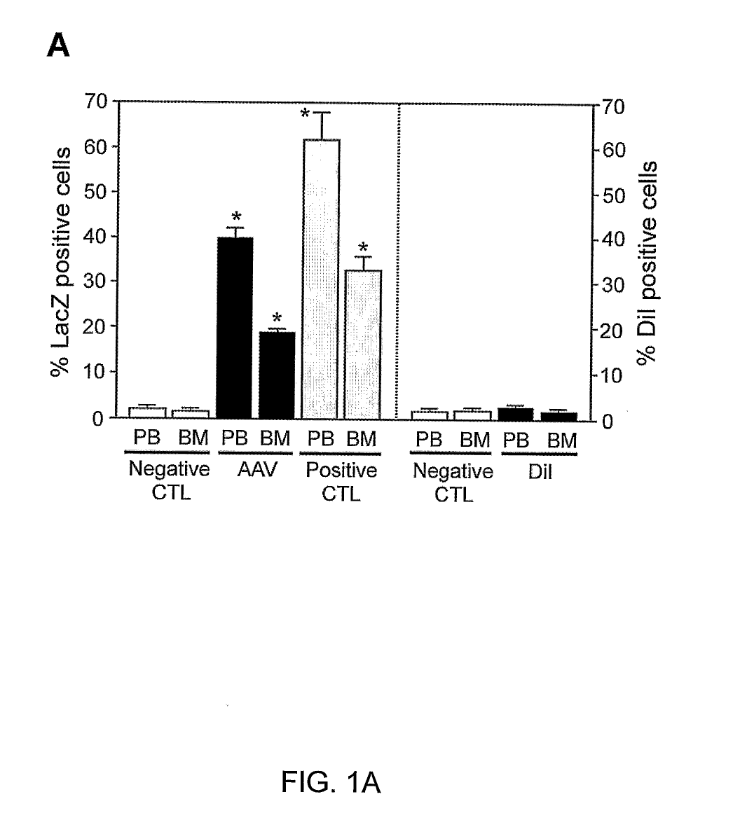

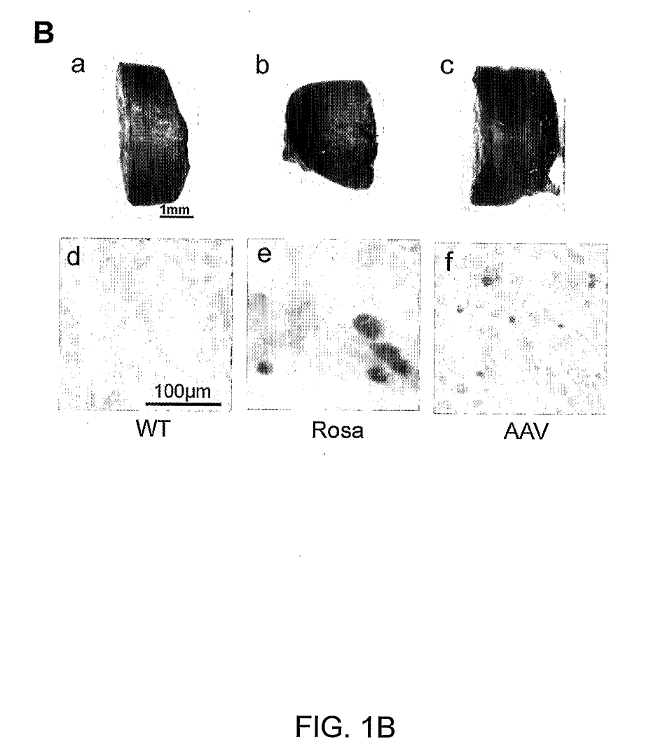

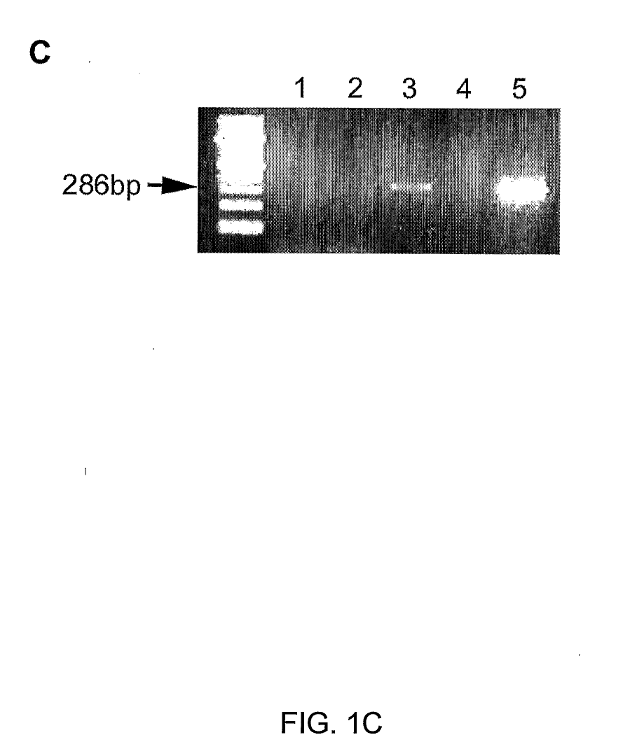

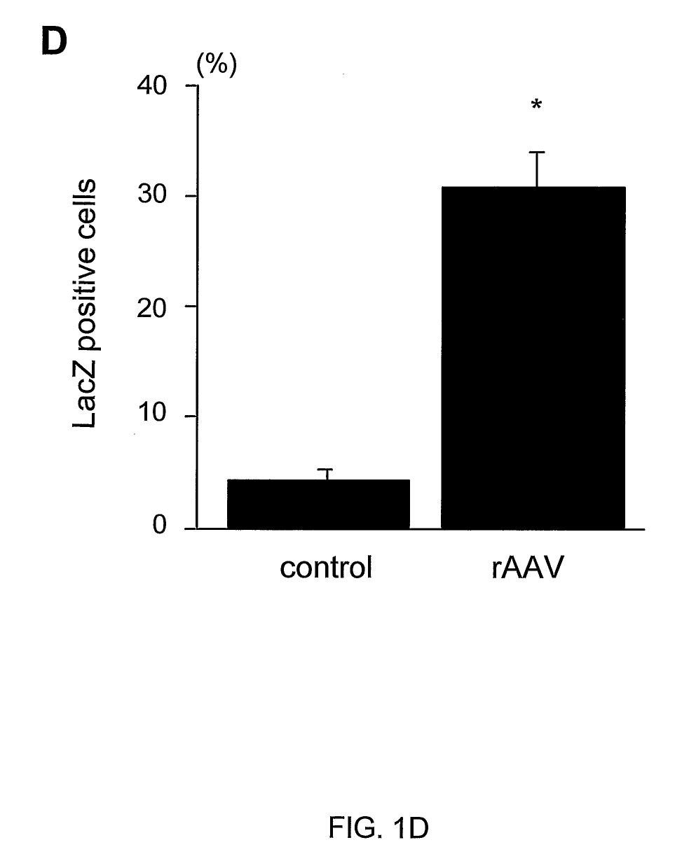

FIG. 1A shows FACS analysis of peripheral blood (PB) and bone marrow (BM) cells harvested 4 weeks after BMT. The results demonstrate stable β-gal expression (40% lacZ positive cells in PB and 20% in BM), as seen in transgenic Rosa mice that constitutively overexpress β-galactosidase [16]. In contrast, the DiI-labeling group displayed only faint signal in both PB and BM at 4 weeks after BMT when those cells were analyzed with DiI specific probes. These results suggest that DiI-labeling is not suitable as a long-term marker of BM-derived cells (more than four weeks). Results shown in FIG. 1A represent the means±S.E. from 6 independent experiments. Asterisk (*) indicates p<0.01 versus control.

We examined the migration of rAAV-labeled BM cells to spleen after BMT. The non-labeled BMT group did not show β-gal positive cells in the spleen either macroscopically or histologically. By contrast, β-gal positive expression was observed both at the tissue level and cellular level in the spleen of rAAV-labeled BMT mice (FIG. 1B). More specifically, FIG. 1B (a-c) illustrate the macroscopic appearance of the spleens of control (a), Rosa mice (b), and rAAV-infected BMT mice (c). The corresponding microscopic images are seen in FIG. 1B (d-f), respectively. The results show that the degree of β-gal staining in rAAV-infected BMT mice was similar to that observed in Rosa mice (compare, for example, FIG. 1Bb with FIG. 1Bc, and FIG. 1Be with FIG. 1Bf).

Because it has been shown that BM of recipient mice is reconstituted with transplanted BM about 3 weeks after BMT [15], we performed a PCR assay 4 weeks after BMT to examine for the presence of rAAV genome in BM. Total DNA was isolated from the BM of each mouse. Referring to FIG. 1C, rAAV sequence was confirmed by PCR to be present only in rAAV-BMT mice but not in controls. More specifically, FIG. 1C shows PCR amplification of rAAV-CMV-lacZ genome sequence from infected BM cells at 1 month after BMT. The primers are designed for a 286-bp fragment from CMV promoter to the lacZ transgene region. The lanes of the gel shown in FIG. 1C are loaded as follows: Lane 1, non-rAAV-infected BM; Lane 2, Rosa mice BM; Lane 3, rAAV-infected BM; Lane 4, negative control; Lane 5, positive control.

Referring now to FIG. 1D, when we infected with rAAV and cultured BM cells for 4 weeks in vitro, we detected about 30.7% lacZ positive cells in the rAAV-infected group compared to less than 5% in the control group. In the results shown in FIG. 1D, n=4, and the asterisk indicates p<0.01 versus control. It has been reported that more than 5 days of cytokine-treatment is required for good transduction of BM cells using lentiviral vectors [5]. In contrast and advantageously, rAAV are easy to use with BM cells, demonstrating rapid infection without any pretreatment with cytokines.

### Example 3

### rAAV-Transduced BM Cells Respond To Cytokines and Ischemic Stress

We have reported that vascular endothelial growth factor (VEGF) promotes mobilization and differentiation of bone marrow-derived mononuclear cell population [13, 14]. Accordingly, we treated recipient mice with VEGF protein and analyzed the kinetics of mobilization of BM cells by FACS analysis.

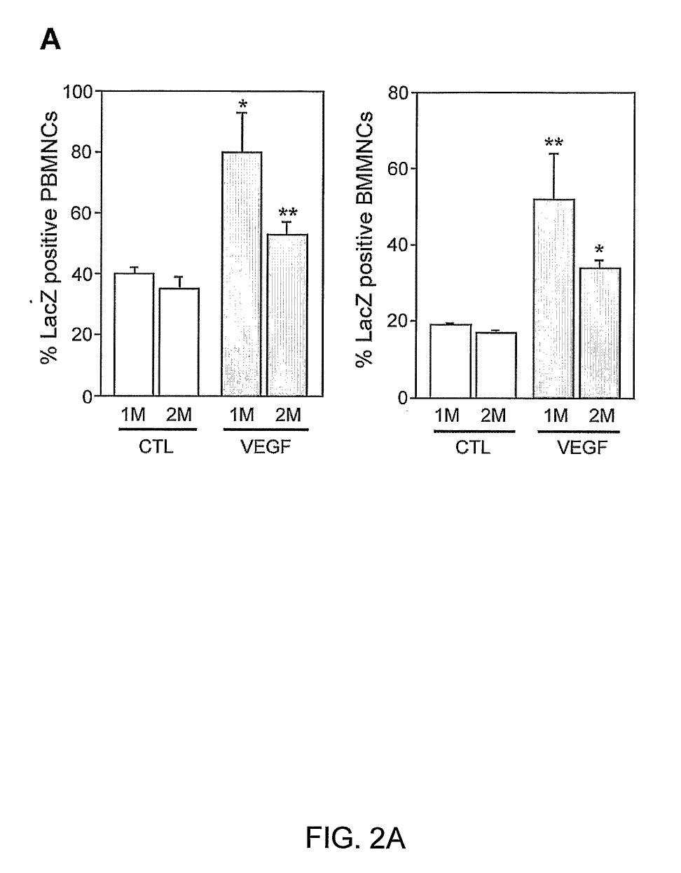

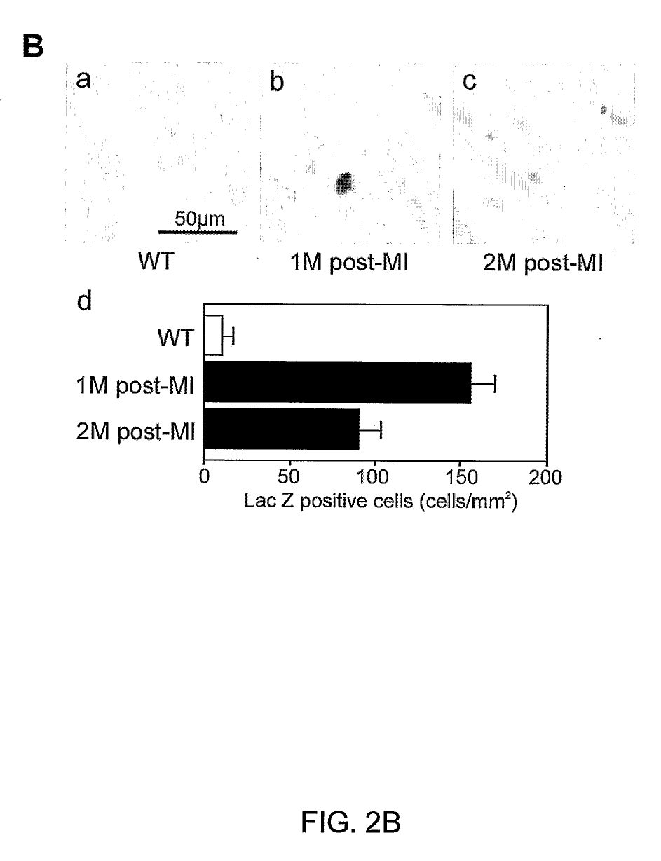

FIG. 2A shows a FACS analysis of PB and BM in BMT mice, with or without treatment with VEGF. The results show that intraperitoneal administration of VEGF protein (100 ng) for 3 consecutive days significantly increased β-gal expression in both PB and BM of recipient mice, even 2 months after BMT. The means±S.E. from 9 independent experiments are presented. *, p<0.01, **, p<0.05 versus control BMT (FIG. 2A).

Finally, when we induced a myocardial infarction (MI) in recipient mice, histological analysis exhibited significant numbers of blue (LacZ positive) cells in the ischemic area of the heart, compared to the non-infected BMT group (FIG. 2B). More specifically, FIG. 2B shows 1-gal expression post myocardial infarction in the hearts of rAAV-labeled BMT mice in the following groups: (a) control; (b) 1 month post-MI; (c) 2 months post-MI. LacZ-positive cells are seen at both 1 and 2 months post-MI (FIG. 2Bb,c). FIG. 2B(d) is a graph showing quantitation of lacZ positive cells in the infracted area of the infected BMT groups at 1 and 2 months post-MI, and in wild type control mice. Control mice were subjected to MI as needed.

These kinetics studies demonstrate that rAAV-infected BM cells can be mobilized and recruited by a BM cell trafficking factor (in this case a cytokine), or by ischemic stress in the same manner as non-infected BM cells. Thus the rAAV vectors of the present invention can be utilized to analyze cell population and mobilization. Our results show that rAAV-transduced BM cells were sustained for 2 months post BMT (FIG. 2A, 2B). In some experiments we observed very low expression at 3 months. In comparison, Tan et al [11] demonstrated that rAAV vectors had long-term transduction capability in hematopoietic stem cells of more than 10 months. In that case, Sca-1⁺ and lin⁻ hematopoietic stem cells were selected and then infected with rAAV vectors.

Without intending to be bound by theory, it is noted that BM cells include many different cell lineages. It is possible that rAAV infectious efficiency might be reduced for mononuclear cells. To determine whether the lacZ positive cells were derived from myeloid cells or lymphoid cells, we performed FACS analysis of bone marrow and peripheral blood using anti-CD11 antibody as a representative myeloid cell lineage marker, and anti-CD4 antibody for the lymphoid cell lineage, 4 weeks after BMT with rAAV infection. We observed that 8% of CD4⁺ cells and 44% of CD11⁺ cells in the BM expressed the lacZ gene. In peripheral blood (PB), 24% of CD4⁺ cells and 70% of CD11⁺ cells expressed the transgene. These results suggest that the rAAV vectors significantly transduced both myeloid cells and lymphoid cells. The ratio of the myeloid cell lineage was higher than the lymphoid cell lineage in our system.

Standard serotype 2 rAAV transduced BM cells well. Collectively, these findings demonstrate that rAAV vectors can be used for labeling bone marrow cells ex vivo prior to transplantation. It is contemplated that use of rAAV vectors to transduce BM cells can represent an efficient and safe means of genetic modification of BM cells for therapeutic purposes including analysis of engraftment dynamics after BMT.

### Example 4

### rAAV Vectors Comprising an IGF-1, an Angiogenic Factor

This example provides methods for making a rAAV vector comprising a human IGF-1 cDNA insert. The NCBI database was searched to obtain DNA sequence information encoding human IGF-1 cDNA. Human IGF-1 cDNA, as defined in NCBI-X00173, was amplified by a standard polymerase chain reaction method using IGF-1 specific PCR primers as shown, using a human gene clone (clone ID: 984882, Catalog# 97002RG, Invitrogen™ life technologies).

### PCR Primers for Amplifying Human IGF-1 Fragment:

(SEQ ID NO: 3)

IGF-1 5′ primer: 5′-CCGAATTCTTCAGAAGCAATGGGA-3′
GAATTC; EcoR1 site

(SEQ ID NO: 4)

IGF-1 3′ primer: 5′-CGGGATCCGTCTTCCTACATCCTG-3′
GGATCC; BamH1 site

The fragment was inserted into a pAAV-CMV vector plasmid as described above. The nucleotide sequence of the IGF-1 insert and the complete sequence of the novel pAAV-CMV-IGF-1 vector are shown below.

Nucleotide sequence of the human IGF-1 cDNA inserted into pAAV-CMV vector plasmid (insert site is designated by two “****” markers):

(SEQ ID NO: 5)

CTTCAGAAGCAATGGGAAAAATCAGCAGTCTTCCAACCCAATTATTTAAG
TGCTGCTTTTGTGATTTCTTGAAGGTGAAGATGCACACCATGTCCTCCTC
GCATCTCTTCTACCTGGCGCTGTGCCTGCTCACCTTCACCAGCTCTGCCA
CGGCTGGACCGGAGACGCTCTGCGGGGCTGAGCTGGTGGATGCTCTTCAG
TTCGTGTGTGGAGACAGGGGCTTTTATTTCAACAAGCCCACACGGTATGG
CTCCAGCAGTCGGAGGGCGCCTCAGACAGGTATCGTGGATGAGTGCTGCT
TCCGGAGCTGTGATCTAAGGAGGCTGGAGATGTATTGCGCACCCCTCAAG
CCTGCCAAGTCAGCTCGCTCTGTCCGTGCCCAGCGCCACACCGACATGCC
CAAGACCCAGAAGGAAGTACATTTGAAGAACGCAAGTAGAGGGAGTGCAG
GAAACAAGAACTACAGGATGTAGGAAGAC

Complete Sequence of the pAAV-CMV-IGF-1 Vector:

(SEQ ID NO: 6)
CAGCAGCTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGAGGGAGT
GGCCAACTCCATCACTAGGGGTTCCTTGTAGTTAATGATTAACCCGCCATGCTAC
TTATCTACGTACATTTATATTGGCTCATGTCCAACATTACCGCCATGTTGACATT
GATTATTGACTAGTTATTAATAGTAATCAATTACGGGGTCATTAGTTCATAGCCC
ATATATGGAGTTCCGCGTTACATAACTTACGGTAAATGGCCCGCCTGGCTGACCG
CCCAACGACCCCCGCCCATTGACGTCAATAATGACGTATGTTCCCATAGTAACGC
CAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAAT
GACGGTAAATGGCCCGCCTGGCATTATGCCCAGTACATGACCTTATGGGACTTTC
CTACTTGGCAGTACATCTACGTATTAGTCATCGCTATTACCATGGTGATGCGGTT
TTGGCAGTACATCAATGGGCGTGGATAGCGGTTTGACTCACGGGGATTTCCAAGT
CTCCACCCCATTGACGTCAATGGGAGTTTGTTTTGGCACCAAAATCAACGGGACT
TTCCAAAATGTCGTAACAACTCCGCCCCATTGACGCAAATGGGCGGTAGGCGTGT
ACGGTGGGAGGTCTATATAAGCAGAGCTCGTTTAGTGAACCGTCAGATCGCCTGG
AGACGCCATCCACGCTGTTTTGACCTCCATAGAAGACACCGGGACCGATCCAGCC
TCCCCTCGAAGCTGATCCTGAGAACTTCAGGGTGAGTCTATGGGACCCTTGATGT
TTTCTTTCCCCTTCTTTTCTATGGTTAAGTTCATGTCATAGGAAGGGGAGAAGTA
ACAGGGTACACATATTGACCAAATCAGGGTAATTTTGCATTTGTAATTTTAAAAA
ATGCTTTCTTCTTTTAATATACTTTTTTGTTTATCTTATTTCTAATACTTTCCCT
AATCTCTTTCTTTCAGGGCAATAATGATACAATGTATCATGCCTCTTTGCACCAT
TCTAAAGAATAACAGTGATAATTTCTGGGTTAAGGCAATAGCAATATTTCTGCAT
ATAAATATTTCTGCATATAAATTGTAACTGATGTAAGAGGTTTCATATTGCTAAT
AGCAGCTACAATCCAGCTACCATTCTGCTTTTATTTTATGGTTGGGATAAGGCTG
GATTATTCTGAGTCCAAGCTAGGCCCTTTTGCTAATCATGTTCATACCTCTTATC
TTCCTCCCACAGCTCCTGGGCAACGTGCTGGTCTGTGTGCTGGCCCATCACTTTG
GCAAA*****GAATTCTTCAGAAGCAATGGGAAAAATCAGCAGTCTTCCAACCCA
ATTATTTAAGTGCTGCTTTTGTGATTTCTTGAAGGTGAAGATGCACACCATGTCC
TCCTCGCATCTCTTCTACCTGGCGCTGTGCCTGCTCACCTTCACCAGCTCTGCCA
CGGCTGGACCGGAGACGCTCTGCGGGGCTGAGCTGGTGGATGCTCTTCAGTTCGT
GTGTGGAGACAGGGGCTTTTATTTCAACAAGCCCACAGGGTATGGCTCCAGCAGT
CGGAGGGCGCCTCAGACAGGTATCGTGGATGAGTGCTGCTTCCGGAGCTGTGATC
TAAGGAGGCTGGAGATGTATTGCGCACCCCTCAAGCCTGCCAAGTCAGCTCGCTC
TGTCCGTGCCCAGCGCCACACCGACATGCCCAAGACCCAGAAGGAAGTACATTTG
AAGAACGCAAGTAGAGGGAGTGCAGGAAACAAGAACTACAGGATGTAGGAAGACG
GATCC******AAGCTGATCTAATTCACCCCACCAGTGCAGGCTGCCTATCAGAA
AGTGGTGGCTGGTGTGGCTAATGCCCTGGCCCACAAGTATCACTAAGCTCGCTTT
CTTGCTGTCCAATTTCTATTAAAGGTTCCTTTGTTCCCTAAGTCCAACTACTAAA
CTGGGGGATATTATGAAGGGCCTTGAGCATCTGGATTCTGCCTAATAAAAAACAT
TTATTTTCATTGCAATGATGTATTTAAATTATTTCTGAATATTTTACTAAAAAGG
GAATGTGGGAGGTCAGTGCATTTAAACATAAAGAAATGAAGAGCTAGTTCAAAAC
CTTGGGAAAATACACTATATCTTAAACTCCATGAAAGAAGGTGAGGCTGCAAACA
GCTAATGCACATTGGCAACAGCCCCTGATGCCTATGCCTTATTCATCCCTCAGAA
AAGGATTCAAGTAGAGGCTTGATTTGGAGGTTAAAGTTTTGCTATGCTGTATTTT
ACATTACTTATTGTTTTAGCTGTCCTCATGAATGTCTTTTCACTACCCATTTGCT
TATCCTGCATCTCTCAGCCTTGACTCCACTCAGTTCTCTTGCTTAGAGATACCAC
CTTTCCCCTGAAGTGTTCCTTCCATGTTTTACGGCGAGATGGTTTCTCCTCGCCT
GGCCACTCAGCCTTAGTTGTCTCTGTTGTCTTATAGAGGTCTACTTGAAGAAGGA
AAAACAGGGGGCATGGTTTGACTGTCCTGTGAGCCCTTCTTCCCTGCCTCCCCCA
CTCACAGTGACCCGGAATCCCTCGACATGGCAGTCTAGATCATTCTTGAAGACGA
AAGGGCCTCGTGTAGATAAGTAGCATGGCGGGTTAATCATTAACTACAAGGAACC
CCTAGTGATGGAGTTGGCCACTCCCTCTCTGCGCGCTCGCTCGCTCACTGAGGCC
GGGCGACCAAAGGTCGCCCGACGCCCGGGCTTTGCCCGGGCGGCCTCAGTGAGCG
AGCGAGCGCGCCAGCTGGCGTAATAGCGAAGAGGCCCGCACCGATCGCCCTTCCC
AACAGTTGCGCAGCCTGAATGGCGAATGGCGATTCCGTTGCAATGGCTGGCGGTA
ATATTGTTCTGGATATTACCAGCAAGGCCGATAGTTTGAGTTCTTCTACTCAGGC
AAGTGATGTTATTACTAATCAAAGAAGTATTGCGACAACGGTTAATTTGCGTGAT
GGACAGACTCTTTTACTCGGTGGCCTCACTGATTATAAAAACACTTCTCAGGATT
CTGGCGTACCGTTCCTGTCTAAAATCCCTTTAATCGGCCTCCTGTTTAGCTCCCG
CTCTGATTCTAACGAGGAAAGCACGTTATACGTGCTCGTCAAAGCAACCATAGTA
CGCGCCCTGTAGCGGCGCATTAAGCGCGGCGGGTGTGGTGGTTACGCGCAGCGTG
ACCGCTACACTTGCCAGCGCCCTAGCGCCCGCTCCTTTCGCTTTCTTCCCTTCCT
TTCTCGCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATTAGGGT
GATGGTTCACGTAGTGGGCCATCGCCCTGATAGACGGTTTTTCGCCCTTTGACGT
TGGAGTCCACGTTCTTTAATAGTGGACTCTTGTTCCAAACTGGAACAACACTCAA
CCCTATCTCGGTCTATTCTTTTGATTTATAAGGGATTTTGCCGATTTCGGCCTAT
TGGTTAAAAAATGAGCTGATTTAACAAAAATTTAACGCGAATTTTAACAAAATAT
TAACGTTTACAATTTAAATATTTGCTTATACAATCTTCCTGTTTTTGGGGCTTTT
CTGATTATCAACCGGGGTACATATGATTGACATGCTAGTTTTACGATTACCGTTC
ATCGATTCTCTTGTTTGCTCCAGACTCTCAGGCAATGACCTGATAGCCTTTGTAG
AGACCTCTCAAAAATAGCTACCCTCTCCGGCATGAATTTATCAGCTAGAACGGTT
GAATATCATATTGATGGTGATTTGACTGTCTCCGGCCTTTCTCACCCGTTTGAAT
CTTTACCTACACATTACTCAGGCATTGCATTTAAAATATATGAGGGTTCTAAAAA
TTTTTATCCTTGCGTTGAAATAAAGGCTTCTCCCGCAAAAGTATTACAGGGTCAT
AATGTTTTTGGTACAACCGATTTAGCTTTATGCTCTGAGGCTTTATTGCTTAATT
TTGCTAATTCTTTGCCTTGCCTGTATGATTTATTGGATGTTGGAATCGCCTGATG
CGGTATTTTCTCCTTACGCATCTGTGCGGTATTTCACACCGCATATGGTGCACTC
TCAGTACAATCTGCTCTGATGCCGCATAGTTAAGCCAGCCCCGACACCCGCCAAC
ACCCGCTGACGCGCCCTGACGGGCTTGTCTGCTCCCGGCATCCGCTTACAGACAA
GCTGTGACCGTCTCCGGGAGCTGCATGTGTCAGAGGTTTTCACCGTCATCACCCA
AACGCGCGAGACGAAAGGGCCTCGTGATACGCCTATTTTTATAGGTTAATGTCAT
GATAATAATGGTTTCTTAGACGTCAGGTGGCACTTTTCGGGGAAATGTGCGCGGA
ACCCCTATTTGTTTATTTTTCTAAATACATTCAAATATGTATCCGCTCATGAGAC
AATAACCCTGATAAATGCTTCAATAATATTGAAAAAGGAAGAGTATGAGTATTCA
ACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCAC
GAGTGGGTTACATCGAACTGGATCTCAACAGCGGTAAGATCCTTGAGAGTTTTCG
CCCCGAAGAACGTTTTCCAATGATGAGCACTTTTAAAGTTCTGCTATGTGGCGCG
GTATTATCCCGTATTGACGCCGGGCAAGAGCAACTCGGTCGCCGCATACACTATT
CTCAGAATGACTTGGTTGAGTACTCACCAGTCACAGAAAAGCATCTTACGGATGG
CATGACAGTAAGAGAATTATGCAGTGCTGCCATAACCATGAGTGATAACACTGCG
GCCAACTTACTTCTGACAACGATCGGAGGACCGAAGGAGCTAACCGCTTTTTTGC
ACAACATGGGGGATCATGTAACTCGCCTTGATCGTTGGGAACCGGAGCTGAATGA
AGCCATACCAAACGACGAGCGTGACACCACGATGCCTGTAGCAATGGCAACAACG
TTGCGCAAACTATTAACTGGCGAACTACTTACTCTAGCTTCCCGGCAACAATTAA
TAGACTGGATGGAGGCGGATAAAGTTGCAGGACCACTTCTGCGCTCGGCCCTTCC
GGCTGGCTGGTTTATTGCTGATAAATCTGGAGCCGGTGAGCGTGGGTCTCGCGGT
ATCATTGCAGCACTGGGGCCAGATGGTAAGCCCTCCCGTATCGTAGTTATCTACA
CGACGGGGAGTCAGGCAACTATGGATGAACGAAATAGACAGATCGCTGAGATAGG
TGCCTCACTGATTAAGCATTGGTAACTGTCAGACCAAGTTTACTCATATATACTT
TAGATTGATTTAAAACTTCATTTTTAATTTAAAAGGATCTAGGTGAAGATCCTTT
TTGATAATCTCATGACCAAAATCCCTTAACGTGAGTTTTCGTTCCACTGAGCGTC
AGACCCCGTAGAAAAGATCAAAGGATCTTCTTGAGATCCTTTTTTTCTGCGCGTA
ATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGAT
ACCAAATACTGTCCTTCTAGTGTAGCCGTAGTTAGGCCACCACTTCAAGAACTCT
GTAGCACCGCCTACATACCTCGCTCTGCTAATCCTGTTACCAGTGGCTGCTGCCA
GTGGCGATAAGTCGTGTCTTACCGCGTTGGACTCAAGACGATAGTTACCGGATAA
GGCGCAGCGGTCGGGCTGAACGGGGGGTTCGTGCACACAGCCCAGCTTGGAGCGA
ACGACCTACACCGAACTGAGATACCTACAGCGTGAGCTATGAGAAAGCGCCACGC
TTCCCCAAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGGGGGGC
GGAGCCTATGGAAAAACGCCAGCAACGCGGCCTTTTTACGGTTCCTGGCCTTTTG
CTGGCCTTTTGCTCACATGTTCTTTCCTGCGTTATCCCCTGATTCTGTGGATAAC
CGTATTACCGCCTTTGAGTGAGCTGATACCGCTCGCCGCAGCCGAACGACCGAGC
GCAGCGAGTCAGTGAGCGAGGAAGCGGAAGAGCGCCCAATACGCAAACCGCCTCT
CCCCGCGCGTTGGCCGATTCATTAATG

Disclosure relating to making and using the PAAV-CMV-IGF-1 vector can be found in Aikawa, R. et al. J. Biol. Chem. 277: 18979-18985 (2002).

In some studies an AAV vector plasmid containing a human growth hormone cDNA (rAAV-hGH) was prepared by enzymatic digestion using both EcoR1 and BamH1.

Following production and purification of the vectors, studies were performed in vitro using cardiomyocytes (cardiac muscle cells). The cells were infected using methods described above, with rAAV vectors expressing either a reporter gene (lacZ), IGF-1 or GH. Expression levels of IGF-1 and AKT were determined by real-time PCR.

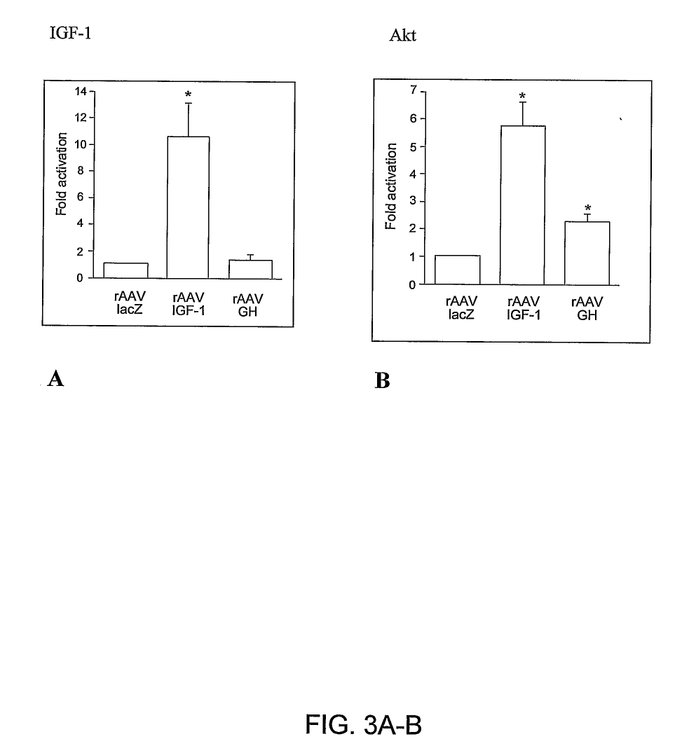

Referring to FIGS. 3A and 3B, results using cardiomycytes infected with the vectors showed that the rAAV-IGF-1 vector markedly increased gene expression of IGF-1, and the downstream effector Akt, compared to rAAV-lacZ control vector. More particularly, FIG. 3 shows real time-PCR analysis of gene expression in the infected cells following RNA preparation and generation of cDNA. The means±S.E. from four independent experiments are shown. *, P<0.05.

### Example 5

### Transduction of Endothelial Progenitor Cells (EPC) with rAAV

There is general recognition that AAV does not transduce endothelial cells well. Furthermore, to our knowledge there has been no previous demonstration of AAV transduction of endothelial progenitor cells (EPC). This Example describes successful transduction of EPC with type 2 rAAV, and methods for optimizing transduction efficiency.

Methods:

EPC Culture and Expansion. Peripheral blood (about 1 ml) was obtained from the hearts of rats and mononuclear cells were isolated by density gradient centrifugation and cultured for 5 days. EPC were expanded ex vivo as described above.

Transduction of EPC. EPC cultures were infected with type 2 AAV-lacZ (also referred to herein as AAV-lacZ(+)) (control) vectors or AAV-IGF-1 vectors, prepared as described above, using approximately 3000 particles/cell. Some cultures were treated for about 1 hour with genistein, a specific inhibitor of cellular protein tyrosine kinases [41], (2-40 μM, preferably about 20 μM). Control vectors used in the genistein experiments also included AAV vector without lacZ, designated AAVlacZ(−).

One week after infection, the cells were analyzed for β-galactosidase activity. AAV-IGF-1 mRNA expression in EPCs was determined by RT-PCR assay using primers specific for human IGF-1 sequences. The primer sequences used in these studies are as shown:

| | |
|---|---|
| 5′-CCATGTCCTCCTCGCATCTC-3′; | (SEQ ID NO: 7) |
| and | |
| 5′-CGTGGCAGAGCTGGTGAAG-3′. | (SEQ ID NO: 8) |

Results:

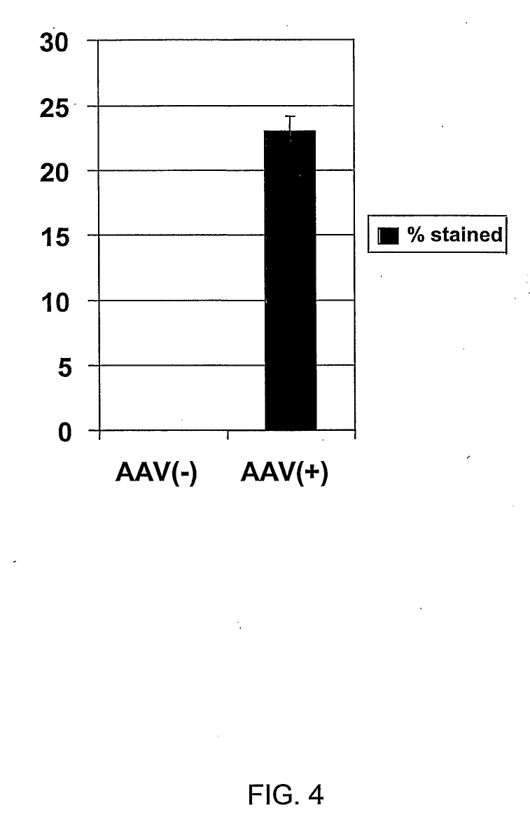

One week after infection with AAV-lacZ, blue-stained spindle-shaped cells exhibiting β-galactosidase (β-gal) activity were abundant in the EPC cultures. Referring to FIG. 4, quantitation of β-gal positive cells showed that 23% (±1.2) of the cultured EPC were successfully transduced and expressed this marker gene from the AAV vector.

To determine if efficacy of AAV expression by EPC could be enhanced, some cultures were pretreated with genistein, as described above. Without intending to be bound by theory, a rationale for this treatment is as follows. The adeno-associated virus 2 (AAV) is a single-stranded DNA-containing human parvovirus. Disadvantageously, the single-stranded nature of the viral genome significantly decreases transduction efficiency because the second-strand viral DNA synthesis is the rate-limiting step. It has been reported that in a human cervical carcinoma cell line (HeLa) transduced with rAAV, a host cell protein, designated the single-stranded D-sequence binding protein (ssD-BP), can specifically and preferentially interact with the single-stranded D sequence within the inverted terminal repeat structure at the 3′ end of the AAV genome, thereby preventing the viral second-strand DNA synthesis [41]. When active, the inhibitory ssD-BP protein is phosphorylated at tyrosine residues. Inhibition of cellular protein tyrosine kinases by genistein, a specific inhibitor of protein tyrosine kinases [42], results in dephosphorylation of the ssD-BP, and is known to significantly augment transgene expression from recombinant AAV in HeLa cells [41].

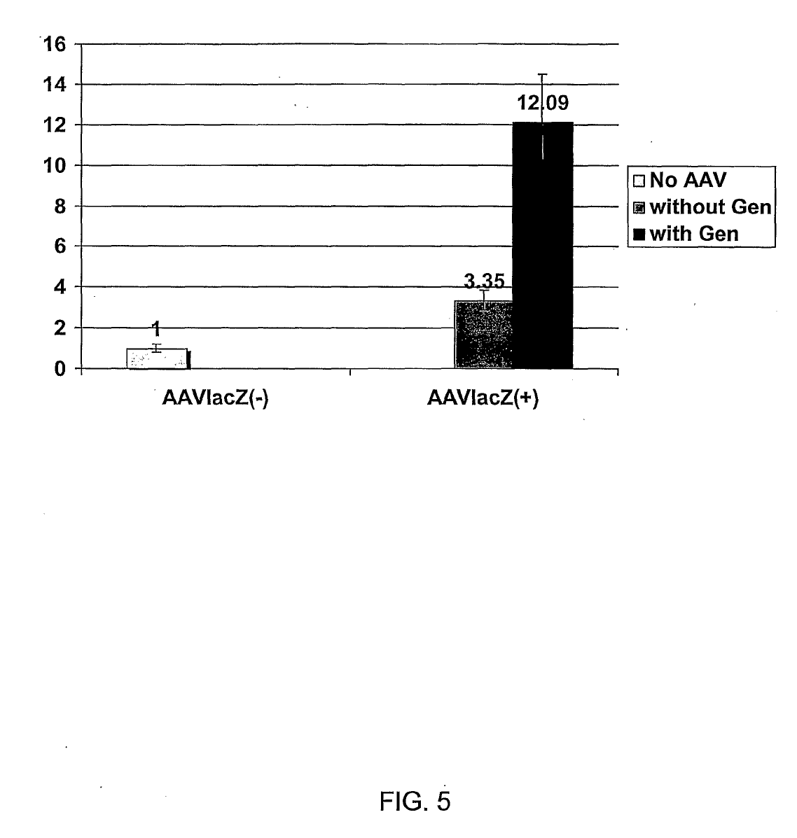

To determine whether efficiency of rAAV transduction of cultured EPC can be enhanced by inhibiting protein tyrosine kinases in these cells, some EPC cultures were pretreated with genestein prior to infection with rAAV, as described above. Results of β-galactosidase assays, performed 14 days after transduction of the cells, are shown in FIG. 5. Referring to FIG. 5, in cultures infected with AAVlacZ(−) control vector, β-gal activity was negligible. In cultures infected with AAVlacZ(+) without or with pretreatment with 20 μM genistein, β-gal activity was 3.35 vs. 12.09, respectively. Thus, lacZ activity was increased approximately four-fold by treating the cells with genistein.

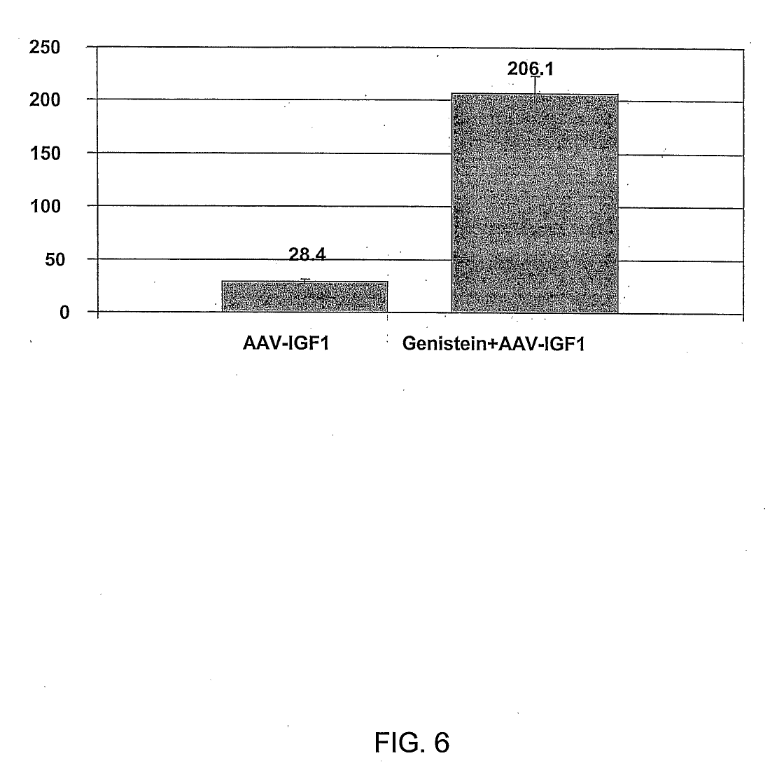

In other experiments, expression of AAV-IGF-1 mRNA in transduced EPCs was determined by RT-PCR assay. Referring to FIG. 6, it is seen that AAV-mediated IGF-1 expression without genistein pretreatment was 28.4 arbitrary units, as compared with 206.1 units following treatment with genistein. Accordingly, by this measure, genistein increased IGF-1 expression from AAV by about seven-fold in EPC.

Results from these studies thus demonstrate that use of an inhibitor of protein tyrosine kinases, such as genistein can significantly augment AAV2-mediated gene expression in transduced EPC.

### Example 6

### Paracrine Effects of IGF-1 on Cardiomyocytes Co-Cultured with AAV-IGF-1-Transduced Endothelial Progenitor Cells (EPC)

Insulin-like growth factor-1 (IGF-1) belongs to the insulin family of peptides and is known to be able to impart several beneficial effects upon cells, providing anti-apoptotic activity as well as promoting proliferation and regeneration of cells. This Example describes a method for providing the beneficial effects of IGF-1 to cardiomyocytes by co-culturing theses cells with EPC transduced with AAV-IGF-1.

Methods:

Co-culture of EPC with Cardiomyocytes. In these studies, EPC transduced with AAV-IGF-1 or AAV-lacZ, prepared as described 5 above, were co-cultured with neonatal rat cardiomyocytes. Briefly, after isolation of the EPC, these cells were added to culture plates of neonatal rat cardiomyocytes at a 1:4 ratio and subsequently cultured for about 7 days.

Nuclear staining was performed using DAPI. EPC were detected by positive immunofluorescent staining with isolectin and DiI. Cardiomyocytes were detected by positive immunofluorescent staining with an antibody specific for α-actinin.

BrdU and TUNEL Assays. To determine paracrine effects of IGF-1 transduced EPC on cardiomyocytes, proliferation of cardiomyocytes was assessed by BrdU assay. The number of proliferating cardiomyocytes in the co-cultures was determined by immunofluorescence microscopy by detecting doubly labeled α-actinin positive cells (red label) showing BrdU positivity (green label). Numbers of cardiomyocytes undergoing apoptosis in these cultures were determined by detecting doubly labeled α-actinin positive cells (red label) showing TUNEL positivity (green label).

Results:

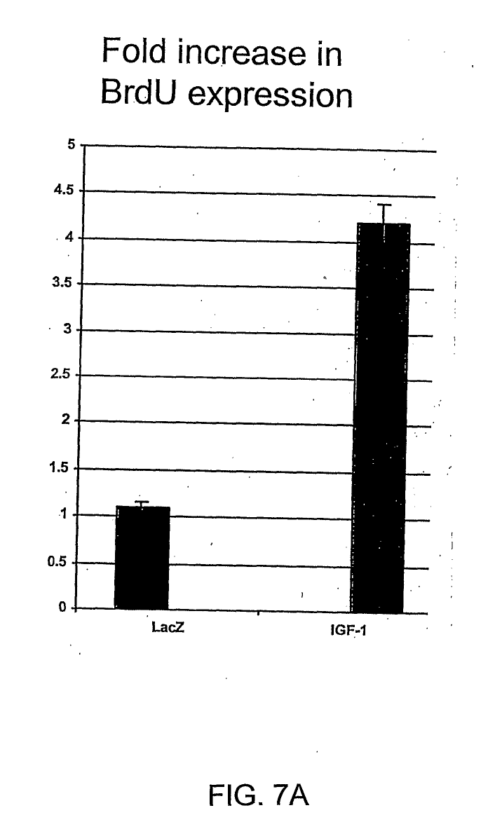

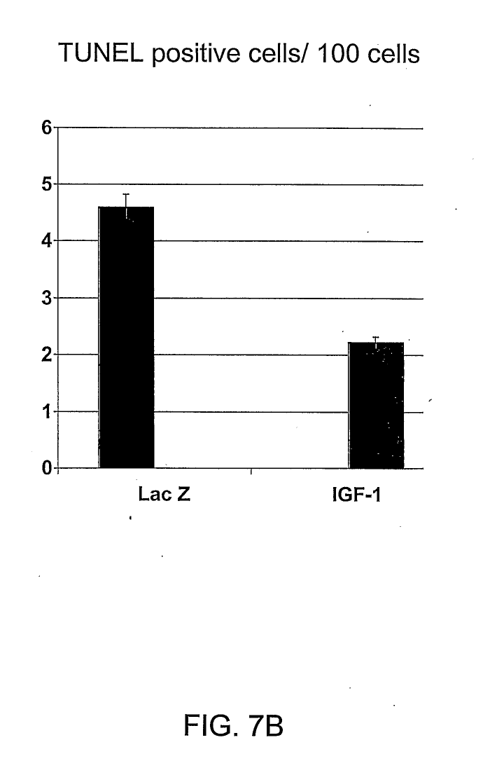

Referring to FIG. 7A, the results showed that rAAV-mediated paracrine IGF-1 secreted from EPC in the cultures stimulated proliferation in the cardiomyocytes in the co-cultures by about four-fold. The presence of the transduced EPC in the cultures also decreased apoptois of the cardiomyocytes. Referring to FIG. 7B, there were 4.3% (±0.2) TUNEL-positive cardiomyocytes in rAAV-lacZ transduced EPC group, compared to 2.1% (±0.1) in rAAV-IGF-1 transduced group.

### Example 7

### Autologous Transplantation of EPC Transduced with AAV-IGF-1 Improves Cardiac Function Following Myocardial Infarction

This Example demonstrates that EPC, when isolated from a mammalian host and expanded and genetically modified with AAV-IGF-1 in vitro, then subsequently transplanted back to the host following myocardial infarct (MI), can significantly improve the host's cardiac function post-MI. At the cellular level, inter alia the cell-based IGF-1 therapy decreases apoptosis and increases proliferation of cardiomyocytes.

Methods:

EPC Isolation, Culture and Transduction with rAAV. Peripheral blood was obtained from the hearts of 5-6 week old Sprague-Dawley rats by puncture of the chest wall and EPC were expanded and cultured essentially as described above. EPC cultures were infected with AAV-IGF-1 or control AAV-lacZ vectors as described.

Approximately one week after obtaining blood, seven host rats were subjected to LAD coronary artery ligation to induce myocardial infarction (MI). Autologous EPC from the hosts transduced with either AAV-IGF-1 or AAV-lacZ (1×10⁴ cells in 100 μl EC-basal medium-2 (Clonetics, San Diego) were administered to five sites in the area affected by MI by intramyocardial injection. Promptly after ligation of the coronary artery, an ischemic area induced by occlusion of the artery was readily visible. AAV-infected EPC were injected into the peri-infarct area immediately following the ligation procedure and the chest wall of the rats was then closed.

Assessment of Treatment. At various times, such as twelve weeks after induction of MI, the effect of the cell-based gene therapy was assessed in the affected areas of the hearts by heart function tests (echocardiography) and immunohistochemical analysis of heart sections. For immunofluorescence microscopy, cell nuclei were stained with DAPI, endothelial cells were detected with endothelial cell specific antibody CD-31, cardiomyocytes were detected with α-actinin antibody, and β-galactosidase activity was detected, as described in Examples above. Cell proliferation was determined using an antibody directed against Ki-67, a specific marker of cellular proliferation.

Results:

Immunohistochemistry. The presence of AAV-transduced EPC in the peri-infarct area of the heart was detected in sections prepared at 12 weeks post-MI, as evidenced by cells that were triply positive for DAPI, CD-31 and β-galactosidase.

To determine if a therapeutic benefit was achieved by transplanting AAV-transduced EPC expressing IGF-1, results were compared from experiments using host rats transplanted with autologous EPC transduced with control (lacZ-AAV) or IGF-1-AAV vectors.

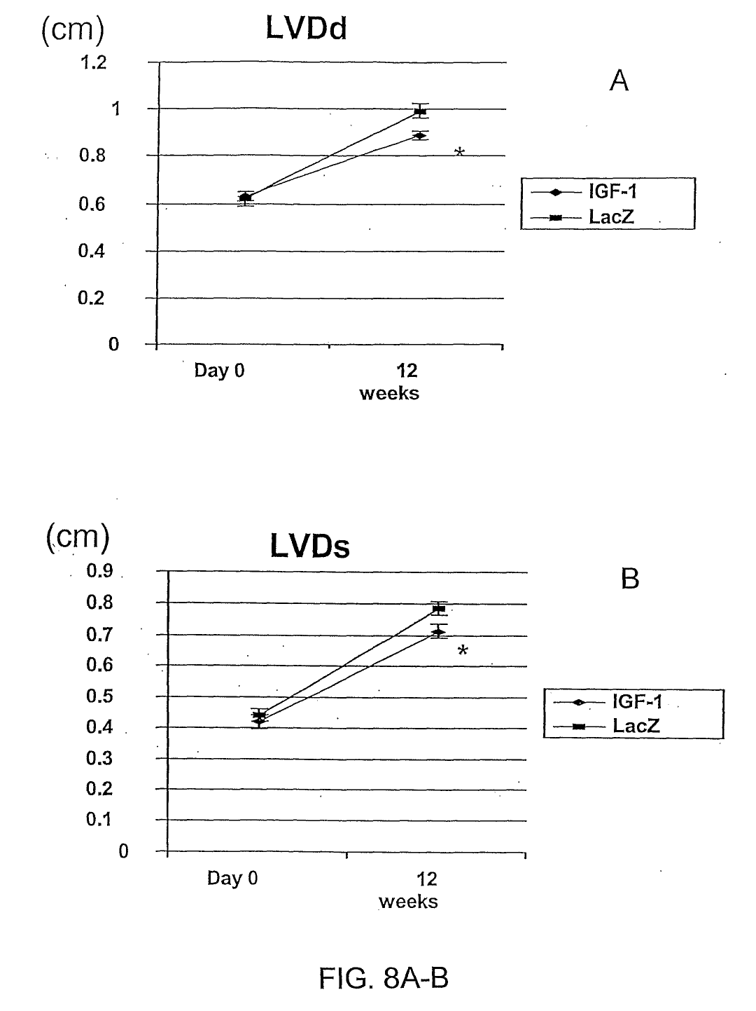

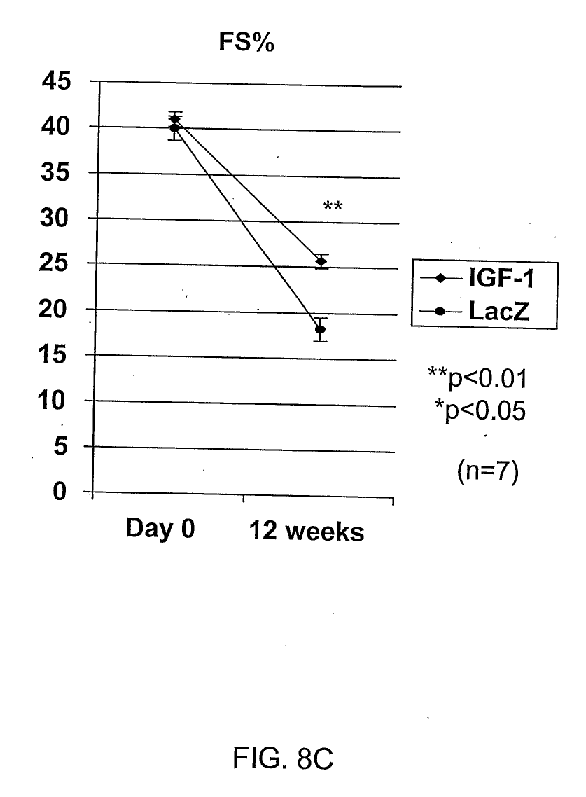

Results of echocardiography studies at 12 weeks after IM are shown in FIGS. 8A-8C. The graphs shown in FIGS. 8A and 8B demonstrate attenuation of adverse ventricular dilatation post-MI in the IGF-1 group, compared with the lacZ group. The graph shown in FIG. 8C illustrates that the IGF-1 transduced group showed significant improvement in the fractional shortening percent (FS %) outcome, compared with the controls. These findings clearly demonstrate that myocardial function was significantly improved in rats receiving intracardiac administration of EPC transduced with AAV-IGF-1, relative to those transplanted with EPC transduced with the control AAV-lacZ vector.

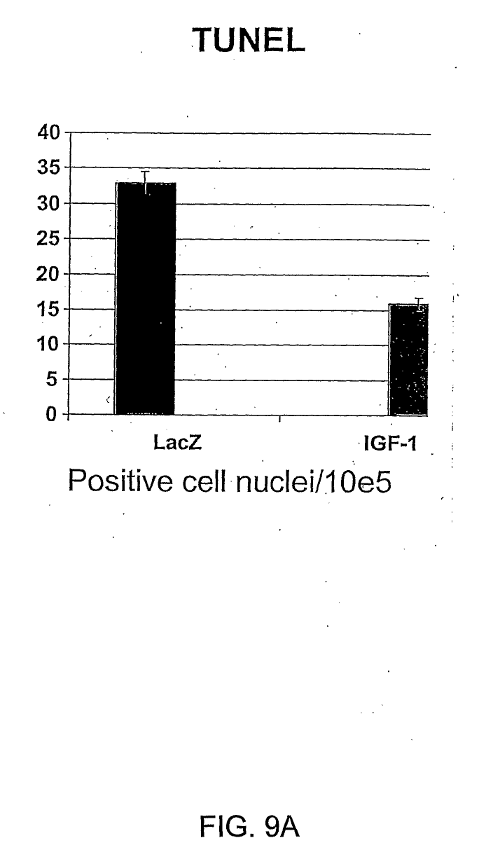

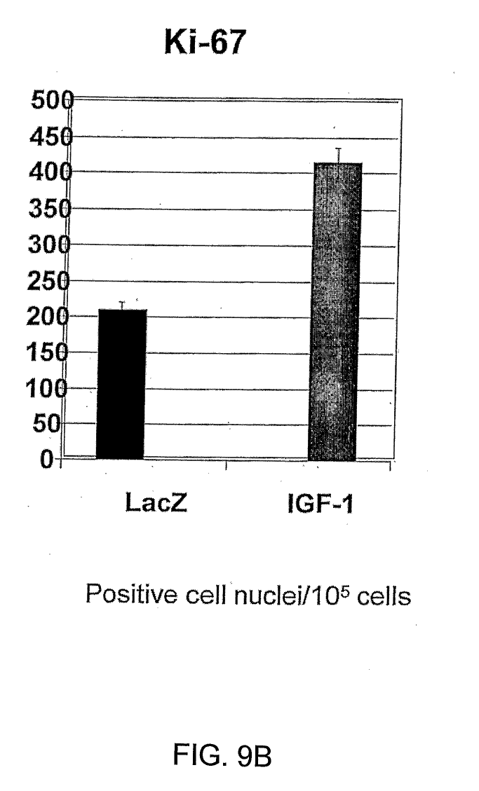

Heart tissues from animals used in this study were processed for several types of analyses and examined microscopically. To determine the apoptotic index in cardiac cells, sections of the hearts were analyzed by fluorescence microscopy, for observation and counting of α-actinin positive cells showing TUNEL positivity. The results, illustrated in FIG. 9A, show that significantly fewer TUNEL-positive cardiac cells were observed in the group transplanted with EPC transduced with IGF-1, as compared with control vector. Thus, cellular gene therapy using EPC to express IGF-1 had an anti-apoptotic effect on cardiac cells in the peri-infarct area following MI.

To assess the effect of transplantation of EPC transduced with IGF-1 on proliferation of cardiac cells in the area impacted by the MI, heart sections as described above were multiply fluorescently stained with DAPI nuclear label, and antibodies against α-actinin and Ki-67 (a marker of cell proliferation). Referring to FIG. 9B, heart tissues from animals transplanted with autologous EPC transduced with AAV-IGF-1 contained significantly more proliferating cardiac cells than those of animals transplanted with autologous EPC transduced with control (AAV-LacZ) vector. Accordingly, transplantation with EPC transduced with IGF-1 had a proliferative effect on cardiomyocytes in the area around the infarct.

Collectively, the results of this Example demonstrate that enhancement of EPC function by AAV-based gene therapy holds great promise for protecting and repairing damaged myocardium, for example by a mechanism that involves using genetically enhanced EPC to deliver of a therapeutic gene, such as IGF-1 directly to a site of tissue injury.

### REFERENCES

It is believed that a review of the references will increase appreciation of the present invention. The following documents are referred to throughout the present disclosure by a number, as indicated below.

[1] Kalka C, Masuda H, Takahashi T, Kalka-Moll W M, Silver M, Kearney M, Li T, Isner J M, Asahara T. Transplantation of ex vivo expanded endothelial progenitor cells for therapeutic neovascularization. Proc Natl Acad Sci USA 2000; 97: 3422-3427.

[2] Kawamoto A, Gwon H C, Iwaguro H, Yamaguchi J I, Uchida S, Masuda H, Silver M, Ma H, Kearney M, Isner J M, Asahara T. Therapeutic potential of ex vivo expanded endothelial progenitor cells for myocardial ischemia. Circulation 2001; 103: 634-637.

[3] Asahara T, Kalka C, Isner J M. Stem cell therapy and gene transfer for regeneration. Gene Ther 2000; 7: 451-457.

[4] Naldini L, Blomer U, Gallay P, Ory D, Mulligan R, Gage F H, Verma I M, Trono D. In vivo gene delivery and stable transduction of nondividing cells by a lentiviral vector. Science 1996; 272: 263-267.

[5] Whartenby K A, Straley E E, Kim H, Racke F, Tanavde V, Gorsi K S, Cheng L, Pardoll D M, Civin C I. Transduction of donor hematopoietic stem-progenitor cells with Fas ligand enhanced short-term engraftment in a murine model of allogeneic bone marrow transplantation. Blood 2002; 100: 3147-3154.

[6] Pathak V K, Temin H M. Broad spectrum of in vivo forward mutations, hypermutations, and mutational hotspots in a retroviral shuttle vector after a single replication cycle: deletions and deletions with insertions. Proc Natl Acad Sci USA 1990; 87: 6024-6028.

[7] Watanabe T, Kuszynski C, Ino K, Heimann D C, Shepard H M, Yasui Y. Maneval D C, Talmadge J E. Gene transfer into human bone marrow hematopoietic cells mediated by adenovirus vectors. Blood 1996; 87: 5032-5039.

[8] Snyder R O, Spratt S K, Lagarde C, Bohl D, Kaspar B, Sloan B, Cohen L K, Danos O. Efficient and stable adeno-associated virus-mediated transduction in the skeletal muscle of adult immunocompetent mice. Hum Gene Ther 1997; 8: 1891-1900.

[9] Aikawa R, Huggins G S, Snyder R O. Cardiomyocyte-specific gene expression following recombinant adeno-associated viral vector transduction. J Biol Chem 2002; 277: 18979-18985.

[10] Melo L C, Agrawal R, Zhang L, Rezvani M, Mangi A A, Ehsan A, Griese D P, Dell'Acqua G. Mann M J, Oyama J, Yet S F, Layne M D, Perrella M A, Dzau V J. Gene therapy strategy for long-term myocardial protection using adeno-associated virus-mediated delivery of heme oxygenase gene. Circulation 2002; 105: 602-607.

[11] Tan M, Qing K, Zhou S, Yoder M C, Srivastava A. Adeno-associated virus 2-mediated transduction and erythroid lineage-restricted long-term expression of the human beta-globin gene in hematopoietic cells from homozygous beta-thalassemic mice. Mol Ther 2001; 3: 940-946.

[12] Chamberlain J R, Schwarze U, Wang P R, Hirata R K, Hankenson K D, Pace J M, Underwood R A, Song K M, Sussman M, Byers P H, Russell D W. Gene targeting in stem cells from individuals with osteogenesis imperfecta. Science 2004; 303: 1198-1201.

[13] Asahara T, Takahashi T, Kalka C, Chen D, Iwaguro H, Inai Y, Silver M, Isner J M. VEGF contributes to postnatal neovascularization by mobilizing bone marrow-derived endothelial progenitor cells. EMBO J. 1999; 18: 39643972.

[14] Kawamoto A, Murayama T, Kusano K, Ii M, Tkebuchava T, Shintani S, Iwakura A, Johnson I, von Samson P, Hanley A, Gavin M, Curry C, Silver M, Ma H, Kearney M, Losordo D W. Synergistic effect of bone marrow mobilization and vascular endothelial growth factor-2 gene therapy in myocardial ischemia. Circulation 2004; 110: 1398-1405.

[15] Iwakura A, Luedemann C, Shastry S, Hanley A, Kearney M, Aikawa R, Isner J M, Asahara T, Losordo D W. Estrogen-mediated, endothelial nitric oxide synthase-dependent mobilization of bone marrow-derived endothelial progenitor cells contributes to reendothelialization after arterial injury. Circulation 2003; 108: 3115-3121.

[16] Zambrowicz B P et al. Disruption of overlapping transcripts in the ROSA βgeo26 gene trapstrain leads to wide spread expression of the β-galactosidase gene in mouse embryos and hematopoietic cells. Proc. Natl. Acad. Sci. 1997; 94(8):3789-3794.

[17] Jiang Y et al. Pluripotency of mesenchymal stem cells derived from adult marrow. Nature 2002; 418:41-49.

[18] Klagsbrun, et al., Annu. Rev. Physiol., 53:217-239 (1991)

[19] Folkman, et al., J. Biol. Chem., 267:10931-10934 (1992)

[20] Symes, et al., Current Opinion in Lipidology, 5:305-312 (1994).

[21] Werner N et al., Arterioscler Thromb Vasc Biol 2002; 22:1567-1572.

[22] Atchison, R. W., Casto, B. C., and Hammon, W. M. (1965) Science 194, 754-756.

[23] Snyder, R. O., Spratt, S. K., Lagarde, C., Bohl, D., Kaspar, B., Sloan, B., Cohen, L. K., and Danos, O. (1997) Hum. Gene Ther 8, 1891-1900

[24] Snyder, R. O., Miao, C. H., Patijn, G. A., Spratt, S. K., Danos, O., Nagy, D., Gown, A. M., Winther, B., Meuse, L., Cohen, L. K., Thompson, A. R., and Kay, M. A. (1997) Nat. Genet. 16, 270-276.

[25] Kaplitt, M. G., Leone, P., Samulski, R. J., Xiao, X., Pfaff, D. W., O'Malley, K. L., and During, M. J. (1994) Nat. Genet. 8, 148-154.

[26] Mandel, R. J., Spratt, S. K., Snyder, R. O., and Leff, S. E. (1997) Proc. Natl. Acad. Sci. U.S.A. 94, 14083-14088.

[27] Svensson, E. C., Marshall, D. J., Woodard, K., Lin, H., Jiang, F., Chu, L., and Leiden, J. M. (1999) Circulation 99, 201-205.

[28] Xiao, X., Li, J., Tsao, Y. P., Dressman, D., Hoffman, E. P., and Watchko, J. F. (2000) J. Virol. 74, 1436-1442.

[29] Walsh, C. E., Nienliuis, A. W., Sainulslki, R. J., Brown, M. G., Miller, J. L., Young, N. S., and Liu, J. M. (1994) J. Clin. Invest. 94, 1440-1448.

[30] Flotte, T., Carter, B., Conrad, C., Guggino, W., Reynolds, T., Rosenstein, B., Taylor, G., Walden, S., and Wetzel, R. (1996) Hum. Gene Ther. 7, 1145-1159.

[31] Su, H., Lu, R., Chang, J. C., and Kan, Y W. (1997) Proc. Natl. Acad. Sci. U.S.A. 94, 13891-13896.

[32] Klein, R. L., Meyer, E. M., Peel, A. L., Zolotukhin, S., Meyers, C., Muzyczka, N., and King, M. A. (1998) Exp Neurol. 150, 183-194.

[33] Jiang, S., Altmann, A., Grimm, D., Kleinschmidt, J. A., Schilling, T., Germann, C., and Haberkorn, U. (2001)Cancer Gene Ther 8, 469-472.

[34] Flannery, J. G., Zolotukhin, S., Vaquero, M. I., La Vail, M. M., Muzyczka, N., and Hauswirth, W. W. (1997) Proc. Natl. Acad. Sci. U.S.A. 94, 6916-6921.

[35] Li, J., Dressman, D., Tsao, Y P., Sakamoto, A., Hoffman, E. P., and Xiao, X. (1999) Gene Ther. 6, 74-82.

[36] Fazio, S., Sabatini, D., Capaldo, B., Vigorito, C., Giordano, A., Guida, R., Pardo, F., Biondi, B., and Sacca, L. (1996) N. Engl. J. Med. 334, 809-814.

[37] Genth-Zotz, S., Zotz, R., Geil, S., Voigtlander, T., Meyer, J., and Darius, H. (1999) Circulation 99, 18-21.

[38] Hongo, M., Ryoke, T., Schoenfeld, J., Hunter, J., Dalton, N., Clark, R., Lowe, D., Chien, K., and Ross, J., Jr. (2000) Basic Res. Cardiol. 95, 431-441.

[39] Mathews, L. C., Gallagher, M. R., Gray, J. T., and Snyder, R. O. (2002) Methods Enzymol. 346, 393-413.

[40] Hardy, S., Kitamura, M., Harris-Stansil, T., Dai, Y, and Phipps, M. L. (1997) J Virol. 71, 1842-1849.

[41] Qing, K., Wang, X.-S., Kube, D. M., Ponnazhagan S., Bajpai, A., Srivastava, A. (1997) Proc. Natl. Acad. Sci. USA 94, 10879-10884.

[42] Akiyama, T., Nakagawa, S., Ogawara, H., Watanabe S., Itoh, N., Shibuya, M., Fukami, Y (1987) J. Biol. Chem. 262:5592-5595.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for expressing a therapeutic or reporter gene in a cardiac tissue or a blood vessel of a host subject, comprising:
(a) contacting a hematopoietic stem cell with a recombinant adeno-associated viral vector comprising a nucleic acid sequence encoding a therapeutic or reporter polypeptide to obtain a transgenic cell stably transduced with the vector; and
(b) administering the cell to a host subject, such that the transgenic cell or a progeny cell thereof populates a cardiac tissue or a blood vessel in the subject and expresses the therapeutic or reporter polypeptide.

2. The method of claim 1, wherein the therapeutic polypeptide is IGF-1, human growth hormone or a functional fragment thereof.

3-6. (canceled)

7. The method of claim 1, wherein the transduced cell is isolated and expanded in vitro to obtain a cell population enriched in bone marrow-derived stem or progenitor cells stably transduced with the vector prior to being administered to the host subject.

8. The method claim 7, wherein IGF-1 is expressed in the cell for at least 8 weeks following administration of the cell to a host subject.

9. The method of claim 1, wherein the cell is selected from the group consisting of a progenitor cell of the bone marrow, a stem cell of the bone marrow, an endothelial progenitor cell, a hematopoietic stem cell (HSC), a mesenchymal stem cell, a multipotent adult progenitor cell, and a human multipotent bone marrow stem cell.

10. The method of claim 9, wherein the cell is an endothelial progenitor cell (EPC).

11. The method of claim 10, wherein the EPC is isolated from bone marrow of a donor subject.

12. The method of claim 10, wherein the EPC is isolated from peripheral blood of a donor subject.

13. The method of claim 1, wherein the cell is treated with genistein prior to infection with an adeno-associated viral vector.

14. The method of claim 1, wherein the cell is a human stem cell having reduced levels of a marker selected from the group consisting of: CD90, CD117, CD34, CD113, FLK-1, tie-2, Oct 4, GATA-4, NKx2.5, Rex-1, CD105, CD117, CD133, MHC class I receptor and MHC class II receptor.

15-24. (canceled)

25. A method for preventing, treating or reducing the severity of a cardiac indication in a host subject in need thereof, the method comprising administering to the subject a therapeutically effective amount of a recombinant cell comprising a recombinant adeno-associated viral vector encoding a IGF-1 or human growth hormone polypeptide or a functional fragment thereof.

26. (canceled)

27. The method of claim 25, wherein the host subject is diagnosed as having a cardiac indication selected from the group consisting of cardiac ischemia, myocardial infarction, cardiomyopathy, cardiomyositis, and heart failure.

28. The method of claim 25, wherein the cell is administered by direct injection into a cardiac tissue or via a blood vessel supplying the heart.

29. The method of claim 25, wherein the cell is treated with genistein prior to infection with the adeno-associated viral vector.

30. A method for reducing apoptosis or increasing proliferation in a cardiac tissue, the method comprising administering to the subject a therapeutically effective amount of a recombinant cell comprising a recombinant adeno-associated viral vector comprising a nucleic acid sequence encoding a IGF-1 or human growth hormone polypeptide or a functional fragment thereof.

31. (canceled)

32. The method of claim 30, wherein the method increases angiogenesis in the cardiac tissue.

33-55. (canceled)

56. A pharmaceutical composition for preventing, treating or reducing the severity of a heart or vascular disorder in a subject in need thereof, the composition comprising a transgenic cell comprising a recombinant adeno-associated viral vector comprising a nucleic acid sequence encoding a IGF-1 or human growth hormone polypeptide or a functional fragment thereof.

57. The pharmaceutical composition of claim 56, wherein the cell is present in a graft.

58. The pharmaceutical composition of claim 56, wherein the composition comprises an additional angiogenic factor or functional fragment thereof.

59. The pharmaceutical composition of claim 56, wherein the composition further comprises a nucleic acid encoding an additional angiogenic factor or functional fragment thereof.

60. The pharmaceutical composition of claim 56, wherein the angiogenic factor is VEGF.

61-62. (canceled)

63. A kit for transducing a hematopoietic stem cell, the kit comprising an adeno-associated viral vector comprising a nucleic acid sequence encoding a IGF-1 or human growth hormone polypeptide or a functional fragment thereof.

64. The kit of claim 63, wherein the kit further comprises directions for administering the vector to a bone marrow derived cell.

65-66. (canceled)

66. (canceled)
